(19) United States
(12) Patent Application Publication
(10) Pub. No.: US 2022/0409646 A1
(43) Pub. Date: Dec. 29, 2022

(54) MALTOSYL-ISOMALTO-OLIGOSACCHARIDES REDUCE SYMPTOMS OF GASTROESOPHAGEAL REFLUX DISEASE

(71) Applicant: ISOThrive Inc., Healdsburg, CA (US)

(72) Inventors: Jack Oswald et al., Healdsburg, CA (US); Lee Madsen, II, Manassas, VA (US); John Selling, Reno, NV (US); Peter Swann, Eden Prairie, MN (US)

(21) Appl. No.: 17/835,727

(22) Filed: Jun. 8, 2022

Related U.S. Application Data
(60) Provisional application No. 63/208,870, filed on Jun. 9, 2021.

(51) Int. Cl. A61K 31/702; A61P 1/04; A61K 45/06

(52) U.S. Cl. CPC A61K 31/702; A61P 1/04; A61K 45/06

(57) ABSTRACT

Compositions and methods are described herein that can reduce the frequency and/or severity of gastroesophageal reflux disease (GERD). Such compositions and methods can include use of maltosyl-isomaltooligosaccharides with or without one or more proton pump inhibitors, one or more potassium-competitive acid blockers, one or more H-2 antagonists, one or more antiacids, one or more bile acid sequestrants, one or more prokinetic agents, one or more dopamine receptor antagonists, one or more coating agents (protectants), one or more antibiotics, one or more probiotics, or a combination thereof.

### CROSS REFERENCE TO RELATED APPLICATIONS

This application claims benefit of priority to the filing date of U.S. Provisional Application Ser. No. 63/208,870, filed Jun. 9, 2021, the contents of which application is specifically incorporated herein by reference in its entireties.

### BACKGROUND

Gastroesophageal reflux disease (GERD) is among the most common diseases in the USA, where 20 percent of the adult population experiences GERD related symptoms weekly. While proton pump inhibitors (PPIs) provide symptom relief for many, about 40 percent of GERD sufferers remain symptomatic. Although PPIs are not approved for chronic use, for want of alternatives, they are prescribed and taken chronically by many who have GERD. Among users, there is concern over potential health risks resulting from long term PPI-induced gastric hypochloridria, including osteoporosis, fractures, pneumonia, C. difficile colitis, small intestinal bacterial overgrowth, hypomagnesemia, anemia, and vitamin B12 deficiency. A documented 46 percent of GERD patients want to stop taking PPIs and 19 percent do so against medical advice.

### SUMMARY

As described herein, the frequency and/or severity of gastroesophageal reflux disease (GERD), non-erosive esophagitis (NERD), functional heartburn, and hypersensitive esophagus can be reduced by taking maltosyl-isomaltooligosaccharides (MIMOs). The maltosyl-isomaltooligosaccharides can, for example, increase the number of heartburn-free days per week by GERD, and have demonstrated efficacy for reducing symptoms associated with non-erosive esophagitis (NERD), functional heartburn, and hypersensitive esophagus sufferers.

Described herein are compositions that can include maltosyl-isomaltooligosaccharides. These compositions can include one or more proton pump inhibitors, one or more potassium-competitive acid blockers (P-CABs), one or more H-2 antagonists, one or more antacids, one or more bile acid sequestrants, one or more prokinetic agents, one or more dopamine receptor antagonists, one or more coating agents (protectants), one or more antibiotics, one or more probiotics, or a combination thereof.

The maltosyl-isomaltooligosaccharide component (ingredient) can have a mass-average molecular weight (Mw) distribution of about 730 to 900 daltons (Da, g/mol), and wherein at least 40% of the maltosyl-isomaltooligosaccharides in the composition have a degree of polymerization (DP) of 3 or more (or a DP of 4 or more, or a DP of 5 or more).

Examples of proton pump inhibitors such as Omeprazole (Prilosec®), Esomeprazole (Nexium®), Lansoprazole (Prevacid®), Rabeprazole (AcipHex®), Pantoprazole (Protonix®), Dexlansoprazole (Dexilant®), Zegerid®, or any combination thereof can be included in the compositions.

Examples of potassium-competitive acid blockers (P-CABs) such as Revaprazan (Revanex), Vonoprazan (Takecab), Tegoprazan, or any combination thereof can be included in the compositions.

Examples of histamine-2 antagonists such as cimetidine (Tagamet), ranitidine (Zantac), famotidine (Pepcid), nizatidine (Axid), or any combination thereof can be included in the compositions.

Examples of antacids such as calcium carbonate (Tums, Alka-Seltzer), magnesium hydroxide (Milk of Magnesia), Mylanta, Maalox, Gelusil, aluminum hydroxide gels (Amphogel, Alternagel), Sodium Bicarbonate, Pepto-bismol, Gaviscon, Riopan, Rolaids, or any combination thereof can be included in the compositions.

Examples of bile acid sequestrants such as cholestyramine (Questran, Prevalite), colestipol (Colestid), colesevelam (Welchol), or any combination thereof can be included in the compositions.

Examples of prokinetic agents such as bethanechol (Urecholine), metoclopramide (Reglan), domperidone (Motilium), cisapride (Propulsid), or any combination thereof can be included in the compositions.

Examples of dopamine receptor antagonists such as domperidone (Motilium), metoclopramide (Reglan), or any combination thereof can be included in the compositions.

Examples of coating agents (protectants) such as sucralfate (Carafate), alginate (Gaviscon), hyaluronic acid plus chondroitin sulfate, or any combination thereof can be included in the compositions.

Examples of antibiotics such as penicillins, cephalosporins, aminoglycosides, fluoroquinolones, carbapenems, tetracyclines, doxycyclines, macrolides, erythromycins, azithromycins, and polymyxins, or any combination thereof can be included in the compositions.

Examples of probiotics such as products containing Lactobacilli, Bifidobacterium, Saccharomyces boulardii or any other useful microorganisms in any combination thereof can be included in the compositions.

Also described herein are methods for treating gastroesophageal reflux disease (GERD), non-erosive esophagitis (NERD), functional heartburn, and hypersensitive esophagus in a human subject. The means of doing so can include administering to the subject, or prescribing for consumption by the subject, a composition that includes maltosyl-isomaltooligosaccharides with a mass average molecular weight distribution of about 730 to 900 daltons, wherein at least 40% of the maltosyl-isomaltooligosaccharides in the composition have a degree of polymerization (DP) of 3 or more, or 4 or more, or 5 or more. The composition can further include one or more proton pump inhibitors, one or more potassium-competitive acid blockers (P-CABs), one or more H-2 antagonists, one or more antacids, one or more bile acid sequestrants, one or more prokinetic agents, one or more dopamine receptor antagonists, one or more coating agents (protectants), one or more antibiotics, one or more probiotics, or a combination thereof.

Such compositions and methods can reduce the symptoms and/or dependence of GERD, non-erosive esophagitis (NERD), functional heartburn, and hypersensitive esophagus sufferers on proton pump inhibitors, potassium-competitive acid blockers (P-CABs), H-2 antagonists, antacids, bile acid sequestrants, prokinetic agents, dopamine receptor antagonists, coating agents (protectants), antibiotics, probiotics, or a combination thereof. For example, when taking the compositions and employing the methods described herein such sufferers can reduce the dosage and frequency of taking proton pump inhibitors, potassium-competitive acid blockers (P-CABs), H-2 antagonists, antacids, bile acid sequestrants, prokinetic agents, dopamine receptor antagonists, coating agents (protectants), antibiotics, probiotics, or a combination thereof.

The methods can involve administering the maltosyl-isomaltooligosaccharides, proton pump inhibitors, potassium-competitive acid blockers (P-CABs), H-2 antagonists, antacids, bile acid sequestrants, prokinetic agents, dopamine receptor antagonists, coating agents (protectants), antibiotics, probiotics, or a combination thereof in a variety of different regimens taken orally, via a nasogastric tube, or via a percutaneous gastric tube. For example, subjects can:

(1) Start taking a proton pump inhibitors, potassium-competitive acid blockers (P-CABs), H-2 antagonists, antacids, bile acid sequestrants, prokinetic agents, dopamine receptor antagonists alone, achieve a baseline of improvement, then start taking maltosyl-isomaltooligosaccharides to increase improvement;

(2) Start taking both maltosyl-isomaltooligosaccharides with one or more proton pump inhibitors, potassium-competitive acid blockers (P-CABs), H-2 antagonists, antacids, bile acid sequestrants, prokinetic agents, dopamine receptor antagonists, or a combination at the same time;

(3) Start taking maltosyl-isomaltooligosaccharides alone, achieve a baseline of improvement, then start one or more proton pump inhibitors, potassium-competitive acid blockers (P-CABs), H-2 antagonists, antacids, bile acid sequestrants, prokinetic agents, dopamine receptor antagonists to increase improvement;

(4) Start as described in either 1, 2 or 3 above, and then stop taking the proton pump inhibitors, potassium-competitive acid blockers (P-CABs), H-2 antagonists, antacids, bile acid sequestrants, prokinetic agents, and dopamine receptor antagonists;

(5) The maltosyl-isomaltooligosaccharides can be taken at night, or at night after brushing and after eating and drinking has ceased;

(6) The maltosyl-isomaltooligosaccharides can be taken in the morning;

(7) The maltosyl-isomaltooligosaccharides can be taken at any time during the day;

(8) The maltosyl-isomaltooligosaccharides can be taken in any of regimens 1-7 above at a dosage of less than 1 gram per dose, 1 gram per dose, more than 1 gram per dose, 2 gram per dose, or more than 2 grams per dose.

Subjects who may achieve only partial relief or no relief on proton pump inhibitors, H-2 antagonists, antacids, bile acid sequestrants, prokinetic agents, dopamine receptor antagonists, coating agents (protectants), antibiotics, bacterial antibiotic peptides, probiotics, and/or on potassium-competitive acid blockers (P-CABs) can add maltosyl-isomaltooligosaccharides to achieve additional relief, or total relief.

### DESCRIPTION OF THE FIGURES

FIG. 1 graphically illustrates results for the Strong Responder Sub-Group D of GERD subjects who received maltosyl-isomaltooligosaccharides (ISOT-101) as described in Examples 1 and 2. The gastrointestinal symptom scores (RQ_GI) as well as the general well-being, sleep disturbances, and other complaints (RQ_WSO) scores are shown over the time of the four week study. As shown, all of these scores significantly improved amongst the strong-responder GERD subjects (Group D). In other words, fewer incidences of acid indigestion and other problems were experienced by these strong-responder GERD subjects when they took maltosyl-isomaltooligosaccharides (ISOT-101) as time progressed during the four-week study.

FIG. 2 graphically illustrates the average scores for all subjects who well-tolerated maltosyl-isomaltooligosaccharides (ISOT-101) administration. As shown, the gastrointestinal symptom scores (RQ_GI) as well as the general well-being, sleep disturbances, and other complaints (RQ_WSO) scores improved amongst all GERD subjects who received maltosyl-isomaltooligosaccharides (ISOT-101). In other words, fewer incidences of acid indigestion and other problems were experienced by all GERD subjects when they took maltosyl-isomaltooligosaccharides (ISOT-101) as time progressed during the four-week study.

FIG. 3A-3D graphically illustrate the change (e.g., reduction) in severity of GERD-related symptoms for subjects receiving MIMOs. FIG. 3A graphically illustrates reduction (change in score, y-axis) in the severity of acid scored by subjects at week four compared to week zero for subjects with differing week zero acid severity scores (x-axis). FIG. 3B graphically illustrates reduction (change in score, y-axis) in the gastrointestinal symptoms (RQ_GI) scored by subjects at week four compared to week zero for subjects with differing week zero gastrointestinal symptom (RQ_GI) scores (x-axis). FIG. 3C graphically illustrates reduction (change in score, y-axis) in the general well-being, sleep disturbances, and other complaints (RQ_WSO) scored by subjects at week four compared to week zero for subjects with differing week zero general well-being, sleep disturbances, and other complaints (RQ_WSO) scores (x-axis). FIG. 3D graphically illustrates reduction (change in score, y-axis) in the of RQ_Total (total) scores scored by subjects at week four compared to week zero for subjects with differing week zero RQ_Total (total) scores (x-axis).

### DETAILED DESCRIPTION

Compositions and methods for treating gastroesophageal reflux disease (GERD), non-erosive esophagitis (NERD), functional heartburn, and hypersensitive esophagus are described herein. The compositions and methods can reduce the dependence of sufferers of GERD, non-erosive esophagitis (NERD), functional heartburn, and hypersensitive esophagus on proton pump inhibitors (PPIs), potassium-competitive acid blockers (P-CABs), H-2 antagonists, antacids, bile acid sequestrants, prokinetic agents, dopamine receptor antagonists, coating agents (protectants), antibiotics, probiotics, or a combination thereof. The compositions and methods include use of maltosyl-isomaltooligosaccharides that can help restore a normal, symptom-free, healthy state to the gastrointestinal tract.

Examples of proton pump inhibitors that can be used in the composition and methods, and where the methods can reduce their administration, include Omeprazole (Prilosec®), Esomeprazole (Nexium®), Lansoprazole (Prevacid®), Rabeprazole (AcipHex®), Pantoprazole (Protonix®), Dexlansoprazole (Dexilant®), Zegerid®, and combinations thereof.

Examples of potassium-competitive acid blockers (P-CABs) that can be used in the composition and methods, and where the methods can reduce their administration, include Revaprazan (Revanex), Vonoprazan (Takecab), Tegoprazan, or any combination thereof.

Examples of histamine-2 antagonists that can be used in the composition and methods, and where the methods can reduce their administration, include cimetidine (Tagamet), ranitidine (Zantac), famotidine (Pepcid), nizatidine (Axid) or any combination thereof.

Examples of antacids that can be used in the composition and methods, and where the methods can reduce their administration, include calcium carbonate (Tums, Alka-Seltzer), magnesium hydroxide (Milk of Magnesia), Mylanta, Maalox, Gelusil, aluminum hydroxide gels (Amphogel, Alternagel), Pepto-bismol, Gaviscon, Rolaids, Riopan, or any combination thereof.

Examples of bile acid sequestrants that can be used in the composition and methods, and where the methods can reduce their administration, include cholestyramine (Questran, Prevalite), colestipol (Colestid), colesevelam (Welchol) or any combination thereof.

Examples of prokinetic agents that can be used in the composition and methods, and where the methods can reduce their administration, include bethanechol (Urecholine), metoclopramide (Reglan), domperidone (Motilium), cisapride (Propulsid) or any combination thereof.

Examples of dopamine receptor antagonists that can be used in the composition and methods, and where the methods can reduce their administration, include domperidone (Motilium), metoclopramide (Reglan), or any combination thereof.

Examples of coating agents (protectants) that can be used in the composition and methods, and where the methods can reduce their administration, include sucralfate (Carafate), alginate (Gaviscon), hyaluronic acid plus chondroitin sulfate, or any combination thereof.

Examples of antibiotics that can be used in the composition and methods, and where the methods can reduce their administration, include penicillins, cephalosporins, sulfonamides, aminoglycosides, fluoroquinolones, carbapenems, tetracyclines, macrolides, erythromycins, azithromycins, bacitracin, colistin, doxycycline, antibiotic cyclic peptides, bacteriocins, and polymyxins, or any combination thereof.

Examples of probiotics that can be used in the composition and methods, and where the methods can reduce their administration, include products containing Lactobacillus, Bifidobacterium, Saccharomyces boulardii or any other useful microorganisms, or any combination thereof.

Results illustrated herein demonstrate that the symptoms and frequency of GERD, non-erosive esophagitis (NERD), functional heartburn, and hypersensitive esophagus can be reduced by maltosyl-isomaltooligosaccharides. Such reduction can reduce or inhibit the progression of such diseases and conditions to more problematic diseases (e.g., Barrett's Esophagus and adenocarcinoma).

When maltosyl-isomaltooligosaccharides (MIMOs) are ingested, the MIMOs can selectively feed probiotic microorganisms rather than potentially harmful bacteria. Some bacteria, including probiotic microorganisms, can naturally produce bacteriocins and defensins that act as antibiotics selective for their natural competitors. Hence, when MIMOs are present, the probiotic microorganisms within the gastrointestinal system can naturally out-compete potentially harmful microorganisms, including various strains of gram negative bacteria. In doing so, maltosyl-isomaltooligosaccharides can treat the root cause of GERD, non-erosive esophagitis (NERD), functional heartburn, and hypersensitive esophagus and inhibit the progression of such diseases and conditions from Barrett's esophagus to adenocarcinoma. The maltosyl-isomaltooligosaccharides therefore curate the microbiome of the distal esophagus and restore a normal, healthy state.

The distal esophageal microbiome differs greatly in subjects with GERD, non-erosive esophagitis (NERD), functional heartburn, and hypersensitive esophagus. The normal microbiome is dominated by gram positive bacteria. The microbiome of patients with GERD, non-erosive esophagitis (NERD), functional heartburn, and hypersensitive esophagus can have abnormal increases in the relative abundance of gram negative organisms that express lipopolysaccharides (LPS) on their outer cell membranes. Toll like receptor four (TLR4), located on the surface of human esophageal epithelial cells, is the receptor for gram negative bacterial LPS. Engagement of TLR4 with LPS has been documented to induce an immune, cytokine mediated inflammatory cascade, which can result in the release of nitric oxide, which relaxes smooth muscle, possibly decreasing lower esophageal sphincter (LES) tone or increasing transient lower esophageal sphincter relaxations. This LES dysfunction, combined with an LPS-related Cox2 induced reduction in gastric emptying, may promote chronic reflux.

Histologically, the inflammation observed with GERD, non-erosive esophagitis (NERD), functional heartburn, and hypersensitive esophagus originates via both acid induced and cytokine induced pathways. Biopsies of areas of erosion exhibit acidic chemical trauma, such as loss of the superficial epithelium and a typical neutrophilic and eosinophilic infiltrate. In non-eroded areas, biopsies also exhibit significant increases in intra-epithelial T cell lymphocytes, widening of intercellular spaces, and basal cell and capillary hyperplasia without surface erosions. Research shows these changes antedate the erosive changes in GERD, non-erosive esophagitis (NERD), functional heartburn, and hypersensitive esophagus. The inflammatory characteristics in GERD, non-erosive esophagitis (NERD), functional heartburn, and hypersensitive esophagus are the same type seen with bacterial inflammation and those seen in patients upon proton pump inhibitor withdrawal. Thus, inflammation and GERD, non-erosive esophagitis (NERD), functional heartburn, and hypersensitive esophagus is likely due both to bacterial dysbiosis as well as to acid induced injury. This type of bacterial dysbiosis remains throughout the disease progression from GERD, non-erosive esophagitis (NERD), functional heartburn, and hypersensitive esophagus to Barret's to esophageal adenocarcinoma. The inflammation is the same as that implicated in gastric adenocarcinoma following infection with H. pylori.

The compositions and methods described herein can reduce the symptoms and/or the frequency of symptoms GERD, non-erosive esophagitis (NERD), functional heartburn, and hypersensitive esophagus. Such symptoms can include sensations of burning in the throat, chest pains, difficulty swallowing, regurgitation, regurgitating acid, sensations of a lump in the throat, epigastric pain, bloating, chronic coughs, laryngitis, asthma, sleep disruption, or combinations thereof.

### Maltosyl-isomaltooligosaccharides (MIMOs)

The maltosyl-isomaltooligosaccharides in the compositions described herein include glucose residues linked mostly by α-(1,6) linkages, and one or two maltose resides. The maltose residues are at the reducing end of the oligosaccharides and are generally linked to a glucose unit by an α-(1,4) linkage. The majority of the linkages between the glucose units in the maltosyl-isomaltooligosaccharides in the compositions are α-(1,6) linkages, although small numbers of α-(1,2), α-(1,3), and α-(1,4) linkages can be present. The maltosyl-isomaltooligosaccharides are therefore typically linear oligosaccharides, with few branch points.

The mass average molecular weight distribution (MWD) of the MIMOs in the compositions described herein can vary depending upon the degree of polymerization (DP). For example, the maltosyl-isomaltooligosaccharides compositions can contain a mass average molecular weight distribution of about 520 to 1200 daltons, or of about 640 to 1000 daltons. In some cases, the maltosyl-isomaltooligosaccharides compositions contain a mass average molecular weight distribution of about 730 to 900 daltons. Hence, the compositions contain maltosyl-isomaltooligosaccharides with a mass average molecular weight distribution of at least 520 daltons, at least 550 daltons, at least 600 daltons, at least 640 daltons, at least 675 daltons, at least 700 daltons, at least 730 daltons, at least 750 daltons, at least 800 daltons, or at least 850 daltons.

The MIMOs in the compositions described herein can have a number of glucose units. For example, the MIMOs in the compositions described herein can have from about 2 to about 18 glucose units, or about 2 to about 17 glucose units, or about 3 to about 16 glucose units, or about 3 to about 15 glucose units, or about 3 to about 14 glucose units, or about 3 to about 13 glucose units, or about 3 to about 12 glucose units. In general, the maltose-containing oligosaccharides have no more than about 17 glucose units, or no more than about 16 glucose units, or no more than about 15 glucose units, or no more than about 14 glucose units, or no more than about 13 glucose units, or no more than about 12 glucose units, or no more than about 11 glucose units, or no more than about 10 glucose units, for example, as detected by HPAEC-PAD or HPLC-RID.

As indicated, the MIMOs in the compositions described herein can have small numbers of α-(1,2), α-(1,3), and α-(1,4) glucosyl linkages. Hence, the MIMOs in the compositions described herein can have small numbers of branch points. For example, the MIMOs in the compositions described herein can have 0-4 branch points, or 0-3 branch points, or 0-2 branch points, or 0-1 branch points.

An example of a structure of a MIMO with a single maltosyl linkage [—O-α-(1,4)-] at the reducing end, and a degree of polymerization (DP) of 5, is shown below:

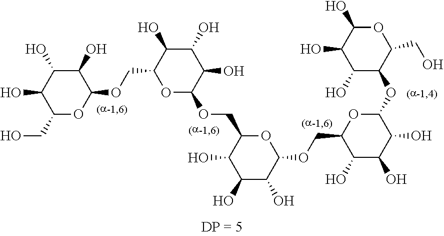 [SMILES: OC[C@H]1O[C@H](OC[C@H]2O[C@H](OC[C@H]3O[C@H](OC[C@H]4O[C@H](O[C@H]5[C@H](O)[C@@H](O)[C@@H](O)O[C@@H]5CO)[C@H](O)[C@@H](O)[C@@H]4O)[C@H](O)[C@@H](O)[C@@H]3O)[C@H](O)[C@@H](O)[C@@H]2O)[C@H](O)[C@@H](O)[C@@H]1O]

The branching pattern and molecular weight can have an effect on the selectivity of a prebiotic composition. For example, Hu et al. (Lett. Appl. Microbiol. 57, pp, 108-114 (2013)) noted that while Lactobacillus reuteri consumed shorter-chain MIMO, the longer chains (higher molecular weight) were preferred by Bifidobacteria. Which bacteria will consume a given prebiotic oligosaccharide depends on the glycolytic enzyme cohort expressed by that bacterial species or bacterial strains.

The compositions described herein (that include MIMOs such as those in ISOThrive™) are prebiotics for a large beneficial (probiotic) class of microorganisms. Examples include Lactobacillus spp., including acidophilus, plantarum, delbrueckii (bulgaricus), rhamnosus, salivarius, fermentum, and reuteri, Bifidobacterium spp., including longum, adolescentis, and bifidum, Streptococcus spp., including thermophilus, salivarius, and the broader viridans (viridans streptococci), Bacillus spp., including subtilis, pumilus, and coagulans.

### Methods of Making Maltosyl-isomaltooligosaccharides

Although MIMOs can be made by any convenient procedure, the MIMOs described herein are typically made under controlled fermentation conditions by strains of bacteria that express dextransucrase (EC 2.4.1.5) enzymes needed to provide the structural attributes of the MIMOs.

In nature, when sucrose is available but in limited amounts, dextransucrases typically make dextran. However, in the presence of sucrose and maltose acceptor molecules, dextransucrases will generate MIMOs. For example, one route to making dextran from glucose by dextransucrase is “interrupted” by the acceptor molecule (e.g., maltose) causing the growth of the chain to terminate prematurely. In effect, the presence of an acceptor such as maltose limits the molecular weight of the oligosaccharide. When this occurs, specifically with maltose, short glucose chains that are terminated with maltose will result. Dextransucrase is therefore a glucosyl transferase that catalyzes the transfer of a glucose residue from sucrose to a growing polyglucan chain.

Dextransucrase (EC 2.4.1.5) producing bacteria can be the primary synthetic “machine” in a fermentation process for production of MIMOs. For example, Leuconostoc mesenteroides NRRL B-512FMC can produce >95% linear α-(1,6) polyglucan from sucrose. The fermentation stoichiometry for aerobic and microaerobic conditions can be as shown below.

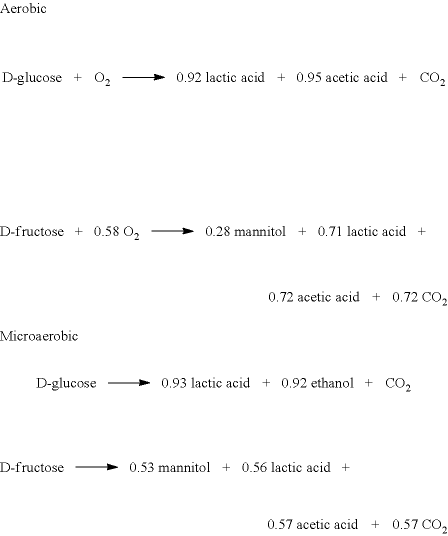

In such microbially-mediated production processes, some of the sucrose is transported into the cell for metabolic purposes, explaining why yield of dextran and or oligosaccharides produced is limited to approximately 50-55%/total sucrose. The competing rates of metabolism and dextran production can impact the outcome of a fermentation. This is one reason that the conditions employed during fermentation are controlled, for example, as described in WO 2016/029198 A1 and WO 2017/127436, both of which are incorporated herein by reference in their entireties.

Useful dextransucrase/alternansucrase (EC 2.4.1.140)/reuteransucrase (EC 2.4.1.; incompletely classified)-producing microorganisms that can be employed to make MIMOs include, but not limited to, Leuconostoc spp (for example, mesenteroides, citreum, gasicomitatum, carnosum, gelidum, inhae, and kimchi), Weissella spp (specifically confusa, kimchi), Lactococcus spp., Streptococcus spp. (specifically mutans), Lactobacillus spp. (e.g. reuteri), Pediococcus spp. (specifically pentosaceus, acidilactici), and certain mutant E. coli.

For example, the MIMO contained in the composition described herein [>80% linear α-(1,6); also called ISOThrive™], can be produced by fermentation using methods described in WO 2016/029198, WO 2019/113446, and WO 2017/127436, both of which are incorporated herein by reference in their entireties.

The inventors have observed that the same enzyme cohort (produced by Leuconostoc mesenteroides subsp. mesenteroides (Tsenkovskii) van Tieghem ATCC® 11449™ or NRRL B-1299) can produce different oligosaccharide branching patterns (e.g., alpha-1,2 branching) when immobilized than when it is native in free solution.

It can be more convenient to produce MIMOs in solution, such as by fermentation. Fermentation can reliably and less expensively produce useful composition of MIMOs.

On a commercial scale, the composition of the present invention may for example be produced as described in U.S. patent application Ser. No. 14/833,094, filed Aug. 22, 2015, entitled “Process for the Production of Isomaltooligosaccharides”, which is herein incorporated by reference in its entirety.

In general, upon start-up of the fermentation process, the entire equipment system is flushed, cleaned and sterilized. A fermentation tank is charged with the requisite media components (typical vitamins, sulfates, phosphates, salts and other materials used for bacterial culture, sucrose, and maltose in a defined sucrose to maltose ratio. All ingredients are non-GMO and certified Kosher/Pareve, including the bacterial vial stock. Separately, the inoculum (for example, ATCC 13146) is grown until achieving an optical density of at least 1 (OD, or absorbance at 660 nm via UV-VIS spectrophotometer), and added to the fermentation in a volume in the range of about 1% to about 10% (1% in the preferred approach) of the amount contained in the bulk media. The fermentation takes place at a temperature of about 27° C. and is maintained at pH 4.0-6.0 (5.5 in the preferred approach) via addition of 50% w/w sodium hydroxide. The fermentation is not aerated, but the headspace is pressurized with air as needed to maintain positive pressure. The fermentation is continued until no fructose is present, for a period of approximately 25 to 60 hours. Most, if not all, of the sucrose and maltose are either consumed or converted to MIMO within a few hours, usually within about 10 hours.

The fermentation is continued for a certain amount of time thereafter to allow the MIMOs produced to be enzymatically rearranged to yield longer chains (higher MWD), if desired. Typically, the time required to create a product, for example, of target MWD of 760-800 Da is about 8-55 Hr. A batch can be run, with periodic sampling and analysis by HPAEC-PAD and/or NMR until the target MWD is reached. The fermentation would then be terminated and moved into downstream processing.

In some cases, the fermentation broth can be manufactured by a method described in in WO 2019/113446 (which is incorporated herein by reference in its entirety). Such a method can include any or all of the following steps:
a. generating a culture medium comprising a sucrose to maltose ratio (S/M) of greater than 2.0, or greater than 2.25, or greater than 2.5, or greater than 2.75, or greater than 2.8, or greater than 2.9, or greater than 3.0, or greater than 3.25, or greater than 3;
b. initiating a fermentation reaction within the culture medium by adding 5% to 15% w/w dextransucrase/alternansucrase-producing microorganisms to the culture medium;
c. conducting fermentation within the culture medium for 10-24 hours to generate a fermentation broth; or a combination thereof.
   The pH of the culture medium during fermentation can be controlled to maintain the pH at about 5.3 to about 5.7, or at about 5.5.

The composition used in the methods may be purified as described in WO 2019/113446 or U.S. patent application Ser. No. 14/833,094, filed Aug. 22, 2015, entitled “Process for the Production of Isomaltooligosaccharides,” which applications are incorporated herein by reference in their entirety.

During the production of the maltosyl-isomaltooligosaccharides composition, the MIMO fermentation can be determined to be complete as determined by the conversion of the fructose to mannitol and by achievement of a target molecular weight distribution. In some cases, the MIMO fermentation is determined to be complete when the fructose is consumed, by conversion of fructose to mannitol, and/or by cessation of the take up of alkali. Hence, the amount of fructose in the final fermentation fluid can be about 4% brix, or less than 7%/brix fructose, or less than 6%/brix fructose, or less than 5%/brix fructose, or less than 4%/brix fructose, or less than 3%/brix fructose, or less than 2%/brix fructose, or less than 1%/brix fructose, or less than 0.5%/brix fructose, or less than 0.25%/brix fructose as detected by HPAEC-PAD or HPLC-RID.

The cells can be separated from the broth by microfiltration, centrifugation, or by other broth clarification processes. The cells are then discarded.

The broth can be concentrated by evaporation to 35-45 brix and can be decolorized with powdered activated carbon (PAC, 0.4-2.0%/broth mass) at about 60-80° C. Alternatively, either granulated activated carbon (GAC) or powdered activated carbon (PAC) may be used, which is later removed by filtration. In another example, granulated activated carbon (GAC) or powdered activated carbon (PAC) can be packed into a jacketed column and used for decolorization in a single-pass or multiple-pass operation.

The decolorized liquor is then demineralized, and the organic acid metabolites removed by a two-stage ion exchange process (TEX), whereby the liquor is contacted first with a strong acid cation (SAC) ion exchange resin, and second, the liquor is contacted with a weak base anion (WBA) TEX resin.

The resulting liquor (2-10 brix) can be adjusted to pH<4.2 (typically 2.0-4.0) with phosphoric acid (H₃PO₄, 85%, 0.25-2.00 kg) and is concentrated by evaporation to 45-60 brix (56 is the preferred approach). The resulting concentrate can be cooled, with gentle agitation to room temperature.

The resulting liquor can be separated from the crystals (D-mannitol) via filtration (Nutsch) or by basket centrifuge. The crystal cake is washed with water (100-200% cake mass at 77% to 95% solids, 125-150% is preferred). The washings are either retained for future batch runs or combined with the liquor and concentrated by evaporation to 60-70 brix (67-69 brix is preferred).

The resulting concentrate can be cooled, with gentle agitation, to room temperature (19-25° C.) and crystals allowed to seed. Once the initial target temperature has been reached, the whole is then cooled slowly to 2-10° C. (5° C. is preferred) until crystallization is complete.

The resulting liquor can be separated from the crystals (D-mannitol) via filtration (Nutsch) or by basket centrifuge. The crystal cake is washed with cold water (100-200% cake mass with 77% to 95% solids, 125-150% cake mass at 95% solids is preferred).

The product liquor can be pasteurized (70° C. for 30 min.) prior to packaging. The washings can be retained and frozen for recycle into the next batch run as appropriate.

Where the concentration of the components in the composition so prepared can fall into the ranges established below. The composition may be liquid at 59-68%/brix or a spray dried/lyophilized powder. The MWD can be inside the range of 730 and 900 Da (mass average).

| | Minimum %/ | Maximum %/ |
|---|---|---|
| Component: | brix: | brix: |
| MIMO DPS-9: | 69.00 | 100.00 |
| D-Mannitol: | 0.00 | 14.00 |
| D-Glucose | 0.00 | 2.50 |
| D-Fructose | 0.00 | 0.50 |
| Sucrose | 0.00 | 6.00 |
| D-Maltose | 0.50 | 7.00 |
| Lactate | 0.00 | 6.50 |
| Glycerol | 0.00 | 2.50 |
| Formate | 0.00 | 1.00 |
| Acetate | 0.00 | 10.00 |
| Purity, %: | 69.00 | 100.00 |

In some cases the manufacturing methods can include:
a. providing a fermentation broth comprising maltosyl-isomaltooligosaccharides, dextransucrase/alternansucrase-producing microorganisms, and culture media;
b. removing the microorganisms from the fermentation broth to produce a cell-free fermentation broth;
c. passing the cell-free fermentation broth through at least one 200 Daltons to 700 Daltons molecular weight cut-off nanofiltration unit to produce a nanofiltered product;
d. passing the nanofiltered product through a strong acid cation ion exchange resin to produce a cation exchange treated product;
e. passing the first ion exchange treated product through a weak base anion ion exchange resin to produce an anion exchange treated product;
f. adjusting the anion exchange treated product pH to a pH less than 5.5 to produce a pH adjusted product;
g. concentrating the pH adjusted product to produce a concentrated MIMO product;
h. filtering the concentrated MIMO product through a microfilter to produce a microfiltered product;
i. pasteurizing the microfiltered product to produce a pasteurized MIMO product; or
a combination thereof.

### Proton Pump Inhibitors

Proton pump inhibitors that can be used in the compositions and methods include Omeprazole (Prilosec®), Esomeprazole (Nexium®), Lansoprazole (Prevacid®), Rabeprazole (AcipHex®), Pantoprazole (Protonix®), Dexlansoprazole (Dexilant®), Zegerid®, and combinations thereof. Administration of maltosyl-isomaltooligosaccharides can reduce the need for such proton pump inhibitors. Hence, when maltosyl-isomaltooligosaccharides are routinely taken, lower dosages of the proton pump inhibitors or less frequent administration of the proton pump inhibitors are possible. For example, when maltosyl-isomaltooligosaccharides are routinely taken, with or without a typical dosage of proton pump inhibitors, the severity and/or frequency of negative gastrointestinal symptoms are reduced.

Omeprazole (Prilosec®) is typically administered at dosages of 20-40 mg daily, but the recommended adult oral dose is 20 mg daily. Although it can be used at any dosage in the compositions and methods described herein, daily use of maltosyl-isomaltooligosaccharides can reduce the dosage or frequency of administering Omeprazole (Prilosec®).

Esomeprazole (Nexium®) is typically administered at dosages of 20-40 mg daily, but the recommended adult oral dose is 20 mg daily. Although it can be used at any dosage in the compositions and methods described herein, daily use of maltosyl-isomaltooligosaccharides can reduce the dosage or frequency of administering Esomeprazole (Nexium®).

Lansoprazole (Prevacid®) is typically administered at dosages of 15-60 mg daily. Although it can be used at any dosage in the compositions and methods described herein, daily use of maltosyl-isomaltooligosaccharides can reduce the dosage or frequency of administering Lansoprazole (Prevacid®).

Pantoprazole (Protonix®) is typically administered at dosages of 20-40 mg daily. Although it can be used at any dosage in the compositions and methods described herein, daily use of maltosyl-isomaltooligosaccharides can reduce the dosage or frequency of administering Pantoprazole (Protonix®).

Dexlansoprazole (Dexilant®) is typically administered at dosages of 30-60 mg daily, but the recommended adult oral dose for GERD is 30 mg daily. Although it can be used at any dosage in the compositions and methods described herein, daily use of maltosyl-isomaltooligosaccharides can reduce the dosage or frequency of administering Dexlansoprazole (Dexilant®).

The compositions and methods described herein that include use of maltosyl-isomaltooligosaccharides can reduce GERD symptoms or the frequency of GERD symptoms. Such GERD symptoms can include sensations of burning in the throat, chest pains, difficulty swallowing, regurgitation, regurgitating acid, sensations of a lump in the throat, chronic coughs, laryngitis, asthma, sleep disruption, or combinations thereof.

When maltosyl-isomaltooligosaccharides are routinely taken, for example daily, the dosage of proton pump inhibitors or the symptoms of GERD can be reduced by at least 10%, at least 20%, at least 30%, at least 40%, at least 50%, at least 60%, at least 70%, at least 80%, at least 90%, or at least 95%.

When maltosyl-isomaltooligosaccharides are routinely taken, for example daily, the frequency of proton pump inhibitor administration and/or the frequency of GERD symptoms can be reduced by at least one day per week, or at least two days per week, or at least three days per week, or at least four days per week, or at least five days per week, or at least six days per week. Hence, when maltosyl-isomaltooligosaccharides are routinely taken, for example daily, subjects suffering from GERD (e.g., who generally take proton pump inhibitors daily) may need to take proton pump inhibitors only six times per week, only five times per week, only four times per week, only three times per week, only two times per week, only once per week, or no times per week.

### Potassium-Competitive Acid Blockers (P-CABs)

Potassium-competitive acid blockers (P-CABs) such as Revaprazan (Revanex® also called YH-1885), Vonoprazan (Takecab®), Tegoprazan, or any combination thereof can be used in the composition and methods described herein. Administration of maltosyl-isomaltooligosaccharides can reduce the need for such potassium-competitive acid blockers (P-CABs). Hence, when maltosyl-isomaltooligosaccharides are routinely taken, lower dosages of the potassium-competitive acid blockers or less frequent administration of the potassium-competitive acid blockers are possible. For example, when maltosyl-isomaltooligosaccharides are routinely taken, with or without a typical dosage of potassium-competitive acid blockers, the severity and/or frequency of negative gastrointestinal symptoms are reduced.

Revaprazan (Revanex®) has been administered at dosages of about 200 mg daily. Although it can be used at any dosage in the compositions and methods described herein, daily use of maltosyl-isomaltooligosaccharides can reduce the dosage or frequency of administering Revaprazan (Revanex®).

Vonoprazan (Takecab®) is typically administered at dosages of 10-20 mg daily; the recommended adult oral dose for GERD is typically 20 mg daily. Although it can be used at any dosage in the compositions and methods described herein, daily use of maltosyl-isomaltooligosaccharides can reduce the dosage or frequency of administering Vonoprazan (Takecab®).

Tegoprazan is typically administered at dosages of 25-100 mg daily; the recommended adult oral dose for GERD is typically 50-100 mg daily. Although it can be used at any dosage in the compositions and methods described herein, daily use of maltosyl-isomaltooligosaccharides can reduce the dosage or frequency of administering Tegoprazan.

The compositions and methods described herein that include use of maltosyl-isomaltooligosaccharides can reduce GERD, non-erosive esophagitis (NERD), functional heartburn, and hypersensitive esophagus, or the symptoms or the frequency thereof. Such symptoms can include sensations of burning in the throat, chest pains, difficulty swallowing, regurgitation, regurgitating acid, sensations of a lump in the throat, chronic coughs, laryngitis, asthma, sleep disruption, or combinations thereof.

When maltosyl-isomaltooligosaccharides are routinely taken, for example daily, the dosage of potassium-competitive acid blockers or the symptoms of GERD can be reduced by at least 10%, at least 20%, at least 30%, at least 40%, at least 50%, at least 60%, at least 70%, at least 80%, at least 90%, or at least 95%.

When maltosyl-isomaltooligosaccharides are routinely taken, for example daily, the frequency of potassium-competitive acid blockers administration and/or the frequency of GERD symptoms can be reduced by at least one day per week, or at least two days per week, or at least three days per week, or at least four days per week, or at least five days per week, or at least six days per week. Hence, when maltosyl-isomaltooligosaccharides are routinely taken, for example daily, subjects suffering from GERD (e.g., who generally take potassium-competitive acid blockers daily) may need to take potassium-competitive acid blockers only six times per week, only five times per week, only four times per week, only three times per week, only two times per week, only once per week, or no times per week.

### H-2 Antagonists

H-2 antagonists such as Famotidine (e.g., Pepcid AC, Pepcid Oral), Cimetidine (e.g., Tagamet, Tagamet HB), Ranitidine (e.g., Zantac, Zantac 75, Zantac Efferdose, Zantac injection, and Zantac Syrup), Nizatidine Capsules (e.g., Axid AR, Axid Capsules, Nizatidine Capsules), or combinations thereof can be used in the compositions and methods described herein. Administration of maltosyl-isomaltooligosaccharides can reduce the need for such H-2 antagonists.

Hence, when maltosyl-isomaltooligosaccharides are routinely taken, lower dosages of the H-2 antagonists or less frequent administration of the H-2 antagonists are possible. For example, when maltosyl-isomaltooligosaccharides are routinely taken, with or without a typical dosage of H-2 antagonists, the severity and/or frequency of negative gastrointestinal symptoms are reduced.

Famotidine (e.g., Pepcid AC, Pepcid Oral) has been administered at dosages of about 20-40 mg once or twice daily. Although it can be used at any dosage in the compositions and methods described herein, daily use of maltosyl-isomaltooligosaccharides can reduce the dosage or frequency of administering Famotidine.

Cimetidine (e.g., Tagamet, Tagamet HB)) has been administered at dosages of about 300 mg one to four times daily. Although it can be used at any dosage in the compositions and methods described herein, daily use of maltosyl-isomaltooligosaccharides can reduce the dosage or frequency of administering Cimetidine.

Ranitidine (e.g., Zantac, Zantac 75, Zantac Efferdose, Zantac injection, and Zantac Syrup) has been administered at dosages of about 75 mg to 300 mg one to two times daily. Although it can be used at any dosage in the compositions and methods described herein, daily use of maltosyl-isomaltooligosaccharides can reduce the dosage or frequency of administering Ranitidine.

Nizatidine (e.g., Axid AR, Axid Capsules, Nizatidine Capsules) has been administered at dosages of about 150 mg to 300 mg one to two times daily. Although it can be used at any dosage in the compositions and methods described herein, daily use of maltosyl-isomaltooligosaccharides can reduce the dosage or frequency of administering Nizatidine.

The compositions and methods described herein that include use of maltosyl-isomaltooligosaccharides can reduce GERD symptoms or the frequency of GERD symptoms. Such GERD symptoms can include sensations of burning in the throat, chest pains, difficulty swallowing, regurgitation, regurgitating acid, sensations of a lump in the throat, chronic coughs, laryngitis, asthma, sleep disruption, or combinations thereof.

When maltosyl-isomaltooligosaccharides are routinely taken, for example daily, the dosage of H-2 antagonists or the symptoms of GERD can be reduced by at least 10%, at least 20%, at least 30%, at least 40%, at least 50%, at least 60%, at least 70%, at least 80%, at least 90%, or at least 95%.

When maltosyl-isomaltooligosaccharides are routinely taken, for example daily, the frequency of H-2 antagonists administration and/or the frequency of GERD symptoms can be reduced by at least one day per week, or at least two days per week, or at least three days per week, or at least four days per week, or at least five days per week, or at least six days per week. Hence, when maltosyl-isomaltooligosaccharides are routinely taken, for example daily, subjects suffering from GERD (e.g., who generally take H-2 antagonists daily) may need to take H-2 antagonists only six times per week, only five times per week, only four times per week, only three times per week, only two times per week, only once per week, or no times per week.

### Antacids

Antacids including calcium carbonate, aluminum hydroxide, magnesium hydroxide, sodium bicarbonate, or combinations thereof can be used in the compositions and methods described herein. Examples of antacids include Gaviscon, Gelusil, Maalox, Mylanta, Riopan, Rolaids, Tums, Alka-Seltzer), Milk of Magnesia, Amphogel, Alternagel, and Pepto-bismol.

Administration of maltosyl-isomaltooligosaccharides can reduce the need for such antacids. Antacids have been administered at dosages of about 300 mg to 4 grams one to four times daily. Although antacids can be used at any dosage in the compositions and methods described herein, daily use of maltosyl-isomaltooligosaccharides can reduce the dosage or frequency of administering antacids.

The compositions and methods described herein that include use of maltosyl-isomaltooligosaccharides can reduce GERD symptoms, the severity of GERD symptoms, or the frequency of GERD symptoms. Such GERD symptoms can include sensations of burning in the throat, chest pains, difficulty swallowing, regurgitation, regurgitating acid, sensations of a lump in the throat, chronic coughs, laryngitis, asthma, sleep disruption, or combinations thereof.

When maltosyl-isomaltooligosaccharides are routinely taken, for example daily, the dosage of antacids or the symptoms of GERD can be reduced by at least 10%, at least 20%, at least 30%, at least 40%, at least 50%, at least 60%, at least 70%, at least 80%, at least 90%, or at least 95%.

When maltosyl-isomaltooligosaccharides are routinely taken, for example daily, the frequency of antacid administration and/or the frequency of GERD symptoms can be reduced by at least one day per week, or at least two days per week, or at least three days per week, or at least four days per week, or at least five days per week, or at least six days per week. Hence, when maltosyl-isomaltooligosaccharides are routinely taken, for example daily, subjects suffering from GERD (e.g., who generally take H-2 antagonists daily) may need to take antacids only six times per week, only five times per week, only four times per week, only three times per week, only two times per week, only once per week, or no times per week.

### Bile Acid Sequestrants

Bile acid sequestrants such as cholestyramine (Questran, Prevalite), colestipol (Colestid), colesevelam (Welchol) or any combination thereof can be used in the compositions and methods described herein. Administration of maltosyl-isomaltooligosaccharides can reduce the need for such bile acid sequestrants.

Hence, when maltosyl-isomaltooligosaccharides are routinely taken, lower dosages of the bile acid sequestrants or less frequent administration of the bile acid sequestrants are possible. For example, when maltosyl-isomaltooligosaccharides are routinely taken, with or without a typical dosage of bile acid sequestrants, the severity and/or frequency of negative gastrointestinal symptoms are reduced.

Cholestyramine (e.g., Questran, Questran Light, Prevalite) has been administered at dosages of about 8 to 24 g daily in divided doses (one to four times daily). Although it can be used at any dosage in the compositions and methods described herein, daily use of maltosyl-isomaltooligosaccharides can reduce the dosage or frequency of administering cholestyramine.

Colestipol (Colestid) has been administered at dosages of 2-16 g daily in divided dosages. Although it can be used at any dosage in the compositions and methods described herein, daily use of maltosyl-isomaltooligosaccharides can reduce the dosage or frequency of administering colestipol.

Colesevelom (Welchol) has been administered at dosages of about 2-4 g daily in divided dosages. Although it can be used at any dosage in the compositions and methods described herein, daily use of maltosyl-isomaltooligosaccharides can reduce the dosage or frequency of administering colesevelom.

The compositions and methods described herein that include use of maltosyl-isomaltooligosaccharides can reduce GERD symptoms or the frequency of GERD symptoms. Such GERD symptoms can include sensations of burning in the throat, chest pains, difficulty swallowing, regurgitation, regurgitating acid, sensations of a lump in the throat, chronic coughs, laryngitis, asthma, sleep disruption, or combinations thereof.

When maltosyl-isomaltooligosaccharides are routinely taken, for example daily, the dosage of bile acid sequestrants or the symptoms of GERD can be reduced by at least 10%, at least 20%, at least 30%, at least 40%, at least 50%, at least 60%, at least 70%, at least 80%, at least 90%, or at least 95%.

When maltosyl-isomaltooligosaccharides are routinely taken, for example daily, the frequency of bile acid sequestrant administration and/or the frequency of GERD symptoms can be reduced by at least one day per week, or at least two days per week, or at least three days per week, or at least four days per week, or at least five days per week, or at least six days per week. Hence, when maltosyl-isomaltooligosaccharides are routinely taken, for example daily, subjects suffering from GERD (e.g., who generally take bile acid sequestrants daily) may need to take bile acid sequestrants only six times per week, only five times per week, only four times per week, only three times per week, only two times per week, only once per week, or no times per week.

### Prokinetic Agents

Prokinetic agents such as bethanechol, metoclopramide, domperidone, cisapride or any combination thereof can be used in the compositions and methods described herein. Administration of maltosyl-isomaltooligosaccharides can reduce the need for such prokinetic agents.

Hence, when maltosyl-isomaltooligosaccharides are routinely taken, lower dosages of the prokinetic agents or less frequent administration of prokinetic agents are possible. For example, when maltosyl-isomaltooligosaccharides are routinely taken, with or without a typical dosage of prokinetic agents, the severity and/or frequency of negative gastrointestinal symptoms are reduced.

Bethanechol (e.g., Urecholine) has been administered at dosages of about 10 to 50 mg one to three times daily. Although it can be used at any dosage in the compositions and methods described herein, daily use of maltosyl-isomaltooligosaccharides can reduce the dosage or frequency of administering bethanechol.

Metoclopromide (e.g., Reglan) has been administered at dosages of 10 to 15 mg one to four times a day. Although it can be used at any dosage in the compositions and methods described herein, daily use of maltosyl-isomaltooligosaccharides can reduce the dosage or frequency of administering metoclopramide.

Domperidone (e.g., Motilium) has been administered at dosages of about 10 to 20 mg three to four times a day. Although it can be used at any dosage in the compositions and methods described herein, daily use of maltosyl-isomaltooligosaccharides can reduce the dosage or frequency of administering domperidone.

Cisapride (e.g., Propulsid) has been administered at dosages of about 10 to 20 mg three to four times a day. Although it can be used at any dosage in the compositions and methods described herein, daily use of maltosyl-isomaltooligosaccharides can reduce the dosage or frequency of administering cisapride.

The compositions and methods described herein that include use of maltosyl-isomaltooligosaccharides can reduce GERD symptoms or the frequency of GERD symptoms. Such GERD symptoms can include sensations of burning in the throat, chest pains, difficulty swallowing, regurgitation, regurgitating acid, sensations of a lump in the throat, chronic coughs, laryngitis, asthma, sleep disruption, or combinations thereof.

When maltosyl-isomaltooligosaccharides are routinely taken, for example daily, the dosage of prokinetic agents or the symptoms of GERD can be reduced by at least 10%, at least 20%, at least 30%, at least 40%, at least 50%, at least 60%, at least 70%, at least 80%, at least 90%, or at least 95%.

When maltosyl-isomaltooligosaccharides are routinely taken, for example daily, the frequency of prokinetic agent administration and/or the frequency of GERD symptoms can be reduced by at least one day per week, or at least two days per week, or at least three days per week, or at least four days per week, or at least five days per week, or at least six days per week. Hence, when maltosyl-isomaltooligosaccharides are routinely taken, for example daily, subjects suffering from GERD (e.g., who generally take prokinetic agents daily) may need to take prokinetic agents only six times per week, only five times per week, only four times per week, only three times per week, only two times per week, only once per week, or no times per week.

### Dopamine Receptor Antagonists

Dopamine receptor antagonists such as metoclopramide and domperidone, or any combination thereof can be used in the compositions and methods described herein. Administration of maltosyl-isomaltooligosaccharides can reduce the need for such dopamine receptor antagonists.

Hence, when maltosyl-isomaltooligosaccharides are routinely taken, lower dosages of dopamine receptor antagonists or less frequent administration of dopamine receptor antagonists are possible. For example, when maltosyl-isomaltooligosaccharides are routinely taken, with or without a typical dosage of dopamine receptor antagonists, the severity and/or frequency of negative gastrointestinal symptoms are reduced.

Metoclopromide (e.g., Reglan) has been administered at dosages of 10 to 15 mg one to four times a day. Although it can be used at any dosage in the compositions and methods described herein, daily use of maltosyl-isomaltooligosaccharides can reduce the dosage or frequency of administering metoclopramide.

Domperidone (e.g., Motilium) has been administered at dosages of about 10 to 20 mg three to four times a day. Although it can be used at any dosage in the compositions and methods described herein, daily use of maltosyl-isomaltooligosaccharides can reduce the dosage or frequency of administering domperidone.

The compositions and methods described herein that include use of maltosyl-isomaltooligosaccharides can reduce GERD symptoms or the frequency of GERD symptoms. Such GERD symptoms can include sensations of burning in the throat, chest pains, difficulty swallowing, regurgitation, regurgitating acid, sensations of a lump in the throat, chronic coughs, laryngitis, asthma, sleep disruption, or combinations thereof.

When maltosyl-isomaltooligosaccharides are routinely taken, for example daily, the dosage of dopamine receptor antagonists or the symptoms of GERD can be reduced by at least 10%, at least 20%, at least 30%, at least 40%, at least 50%, at least 60%, at least 70%, at least 80%, at least 90%, or at least 95%.

When maltosyl-isomaltooligosaccharides are routinely taken, for example daily, the frequency of dopamine receptor antagonist administration and/or the frequency of GERD symptoms can be reduced by at least one day per week, or at least two days per week, or at least three days per week, or at least four days per week, or at least five days per week, or at least six days per week. Hence, when maltosyl-isomaltooligosaccharides are routinely taken, for example daily, subjects suffering from GERD (e.g., who generally take dopamine receptor antagonists daily) may need to take dopamine receptor antagonists only six times per week, only five times per week, only four times per week, only three times per week, only two times per week, only once per week, or no times per week.

### Coating Agents (Protectants)

Coating agents (protectants) such as sucralfate (Carafate), alginate (Gaviscon), hyaluronic acid plus chondroitin sulfate, or any combination thereof can be used in the compositions and methods described herein. Administration of maltosyl-isomaltooligosaccharides can reduce the need for such coating agents (protectants).

Hence, when maltosyl-isomaltooligosaccharides are routinely taken, lower dosages of coating agents (protectants) or less frequent administration of coating agents (protectants) are possible. For example, when maltosyl-isomaltooligosaccharides are routinely taken, with or without a typical dosage of coating agents (protectants), the severity and/or frequency of negative gastrointestinal symptoms are reduced.

Sucralfate (Carafate) has been administered at dosages of 1 g two to four times a day. Although it can be used at any dosage in the compositions and methods described herein, daily use of maltosyl-isomaltooligosaccharides can reduce the dosage or frequency of administering sucralfate.

Alginate (e.g., Gaviscon) has been administered at dosages of about 1 to 2 g three to four times a day. Although it can be used at any dosage in the compositions and methods described herein, daily use of maltosyl-isomaltooligosaccharides can reduce the dosage or frequency of administering alginate.

Hyaluronic Acid plus Chondroitin Sulfate has been administered at dosages of 2 to 8 g daily in divided doses throughout the day. Although it can be used at any dosage in the compositions and methods described herein, daily use of maltosyl-isomaltooligosaccharides can reduce the dosage or frequency of administering hyaluronic acid plus chondroitin sulfate.

The compositions and methods described herein that include use of maltosyl-isomaltooligosaccharides can reduce GERD symptoms or the frequency of GERD symptoms. Such GERD symptoms can include sensations of burning in the throat, chest pains, difficulty swallowing, regurgitation, regurgitating acid, sensations of a lump in the throat, chronic coughs, laryngitis, asthma, sleep disruption, or combinations thereof.

When maltosyl-isomaltooligosaccharides are routinely taken, for example daily, the dosage of coating agents (protectants) or the symptoms of GERD can be reduced by at least 10%, at least 20%, at least 30%, at least 40%, at least 50%, at least 60%, at least 70%, at least 80%, at least 90%, or at least 95%.

When maltosyl-isomaltooligosaccharides are routinely taken, for example daily, the frequency of coating agent (protectants) administration and/or the frequency of GERD symptoms can be reduced by at least one day per week, or at least two days per week, or at least three days per week, or at least four days per week, or at least five days per week, or at least six days per week. Hence, when maltosyl-isomaltooligosaccharides are routinely taken, for example daily, subjects suffering from GERD (e.g., who generally take coating agents (protectants) daily) may need to take coating agents (protectants) only six times per week, only five times per week, only four times per week, only three times per week, only two times per week, only once per week, or no times per week.

### Antibiotics

Antibiotics such as penicillins, cephalosporins, aminoglycosides, sulfonamides, fluoroquinolones, carbapenems, tetracyclines, doxycyclines, macrolides, peptide antibiotics, polypeptide antibiotics, erythromycins, azithromycins, polymyxins, or any combination thereof can be used in the compositions and methods described herein. Administration of maltosyl-isomaltooligosaccharides can reduce the need for such antibiotics.

Hence, when maltosyl-isomaltooligosaccharides are routinely taken, lower dosages of the antibiotics or less frequent administration of antibiotics are possible. For example, when maltosyl-isomaltooligosaccharides are routinely taken, with or without a typical dosage of antibiotics, the severity and/or frequency of negative gastrointestinal symptoms are reduced.

Penicillins (e.g., Ampicillin) have been administered at dosages of about 250 to 500 mg four times daily. Although it can be used at any dosage in the compositions and methods described herein, daily use of maltosyl-isomaltooligosaccharides can reduce the dosage or frequency of administering penicillins.

Cefalosporins (e.g., Ancef) have been administered at dosages of 250 to 500 mg two to four times a day. Although it can be used at any dosage in the compositions and methods described herein, daily use of maltosyl-isomaltooligosaccharides can reduce the dosage or frequency of administering cefalosporins.

Aminoglycosides (e.g., Gentamicin) have been administered at dosages of about 3 to 5 mg/kg/day in divided doses. Although it can be used at any dosage in the compositions and methods described herein, daily use of maltosyl-isomaltooligosaccharides can reduce the dosage or frequency of administering aminoglycosides.

Fluoroquinolones (e.g., Ciprofloxacin) have been administered at dosages of about 250 to 500 mg two times a day. Although it can be used at any dosage in the compositions and methods described herein, daily use of maltosyl-isomaltooligosaccharides can reduce the dosage or frequency of administering flouroquinolones.

Carbapenems (e.g., Imipenem and Primaxin) have been administered at dosages of about 500 mg four times a day. Although it can be used at any dosage in the compositions and methods described herein, daily use of maltosyl-isomaltooligosaccharides can reduce the dosage or frequency of administering carbapenems.

Polymixins (e.g., Colistin) have been administered at dosages of about 4 to 6 mg/kg daily in divided doses. Although it can be used at any dosage in the compositions and methods described herein, daily use of maltosyl-isomaltooligosaccharides can reduce the dosage or frequency of administering polymixins.

Sulfonamides (e.g., ulfadiazine, sulfamethizole (Thiosulfil Forte), sulfamethoxazole (Gantanol), sulfasalazine (Azulfidine), or sulfisoxazole (Gantrisin)) have been administered at dosages of about 1-4 grams one to four times per day. Although it can be used at any dosage in the compositions and methods described herein, daily use of maltosyl-isomaltooligosaccharides can reduce the dosage or frequency of administering sulfanamides.

The compositions and methods described herein that include use of maltosyl-isomaltooligosaccharides can reduce GERD symptoms or the frequency of GERD symptoms. Such GERD symptoms can include sensations of burning in the throat, chest pains, difficulty swallowing, regurgitation, regurgitating acid, sensations of a lump in the throat, chronic coughs, laryngitis, asthma, sleep disruption, or combinations thereof.

When maltosyl-isomaltooligosaccharides are routinely taken, for example daily, antibiotics may not be necessary to reduce the symptoms of GERD or reduce the frequency of GERD.

Hence, when maltosyl-isomaltooligosaccharides are routinely taken, for example daily, subjects suffering from GERD (e.g., who may need antibiotics for GERD) may not need to take antibiotics.

### Probiotics

Probiotic microorganisms such as Lactobacillus, Bifidobacterium, Saccharomyces boulardii, or any other useful bacteria in any combination thereof can be used in the compositions and methods described herein. Administration of maltosyl-isomaltooligosaccharides can reduce the need for such probiotics.

Hence, when maltosyl-isomaltooligosaccharides are routinely taken, lower dosages of probiotics or less frequent administration of probiotics are possible. For example, when maltosyl-isomaltooligosaccharides are routinely taken, with or without a typical dosage of probiotics, the severity and/or frequency of negative gastrointestinal symptoms are reduced.

Probiotics have been administered at dosages of tens of billions of units one to four times a day. Although it can be used at any dosage in the compositions and methods described herein, daily use of maltosyl-isomaltooligosaccharides can reduce the dosage or frequency of administering probiotics.

The compositions and methods described herein that include use of maltosyl-isomaltooligosaccharides can reduce GERD symptoms or the frequency of GERD symptoms. Such GERD symptoms can include sensations of burning in the throat, chest pains, difficulty swallowing, regurgitation, regurgitating acid, sensations of a lump in the throat, chronic coughs, laryngitis, asthma, sleep disruption, or combinations thereof.

When maltosyl-isomaltooligosaccharides are routinely taken, for example daily, the dosage of probiotics or the symptoms of GERD can be reduced by at least 10%, at least 20%, at least 30%, at least 40%, at least 50%, at least 60%, at least 70%, at least 80%, at least 90%, or at least 95%.

When maltosyl-isomaltooligosaccharides are routinely taken, for example daily, the frequency of probiotics administration and/or the frequency of GERD symptoms can be reduced by at least one day per week, or at least two days per week, or at least three days per week, or at least four days per week, or at least five days per week, or at least six days per week. Hence, when maltosyl-isomaltooligosaccharides are routinely taken, for example daily, subjects suffering from GERD (e.g., who generally take probiotics daily) may need to take probiotics only six times per week, only five times per week, only four times per week, only three times per week, only two times per week, only once per week, or no times per week.

### Treatment

The compositions described herein can be administered in a regimen that reduces the symptoms (or severity thereof) or the incidence of GERD, non-erosive esophagitis (NERD), functional heartburn, and hypersensitive esophagus, and/or that reduces the dosage or frequency of administering medications for treating such diseases and conditions. Such regimens can foster the growth and/or activity of probiotic microorganisms that can be present in the digestive or gastrointestinal system of an animal. For example, the compositions described herein can be administered to animals, including humans, domesticated animals, zoo animals, livestock, and wild animals. The compositions can be used routinely or intermittently to foster the growth and/or activity of probiotic microorganisms that can be present in the digestive or gastrointestinal system of an animal.

The compositions contain MIMOs in an amount effective for reducing the symptoms of GERD and/or that reduces the dosage or frequency of administering proton pump inhibitors. Such amounts of MIMOs can foster the growth and/or activity of probiotic microorganisms. An effective amount can, for example, be an amount sufficient to foster probiotic microorganism growth by at least about 5%, 10%, 20%, 25%, 30%, 40%, 50%, 60%, 70%, 75%, 80%, 85%, 90%, 93%, 94%, 95%, 96%, 97%, 98%, 99%, 99.5%, or 99.9%. In some cases, the population of probiotic microorganisms can be increased in a gastrointestinal system of an animal who has received the compositions described herein. For example, the population of probiotic microorganisms can increase by about two-fold, or about three-fold, or about four-fold, or about five-fold, or about six-fold, or about seven-fold, or about ten-fold.

The compositions can include one or more proton pump inhibitors, one or more potassium-competitive acid blockers (P-CABs), one or more H-2 antagonists, one or more antacids, one or more bile acid sequestrants, one or more prokinetic agents, one or more dopamine receptor antagonists, one or more coating agents (protectants), one or more antibiotics, one or more probiotics, or a combination thereof at the same dosage as the recommended dosage, or in amounts less than the typical recommended dose for GERD, non-erosive esophagitis (NERD), functional heartburn, and hypersensitive esophagus.

The proton pump inhibitors that can be used in the compositions and methods can include Omeprazole (Prilosec®), Esomeprazole (Nexium®), Lansoprazole (Prevacid®), Rabeprazole (AcipHex®), Pantoprazole (Protonix®), Dexlansoprazole (Dexilant®), Zegerid®, and combinations thereof.

The compositions and methods can include use of one or more potassium-competitive acid blockers at the same dosage as the recommended dosage, or in amounts less than the typical recommended dose for GERD. The potassium-competitive acid blockers that can be used in the compositions and methods can include Revaprazan (Revanex® also called YH-1885), Vonoprazan (Takecab®), Tegoprazan, or any combination thereof.

The compositions and methods can include use of one or more H-2 antagonists at the same dosage as the recommended dosage, or in amounts less than the typical recommended dose for GERD. The H-2 antagonists that can be used in the compositions and methods can include H-2 antagonists such as Famotidine (e.g., Pepcid AC, Pepcid Oral), Cimetidine (e.g., Tagamet, Tagamet HB), Ranitidine (e.g., Zantac, Zantac 75, Zantac Efferdose, Zantac injection, and Zantac Syrup), Nizatidine Capsules (e.g., Axid AR, Axid Capsules, Nizatidine Capsules), or combinations thereof or any combination thereof.

The compositions and methods can include use of one or more antacids at the same dosage as the recommended dosage, or in amounts less than the typical recommended dose for GERD. The antacids that can be used in the compositions and methods can include calcium carbonate, aluminum hydroxide, magnesium hydroxide, sodium bicarbonate, or combinations thereof. Examples of antacids include Gaviscon, Gelusil, Maalox, Mylanta, Riopan, Rolaids, Tums, Alka-Seltzer, Milk of Magnesia, Amphogel, Alternagel, and Pepto-bismol.

The compositions and methods can include use of one or more bile acid sequestrants at the same dosage as the recommended dosage, or in amounts less than the typical recommended dose for GERD. The bile acid sequestrants that can be used in the compositions and methods can include bile acid sequestrants such as cholestyramine (Questran, Prevalite), colestipol (Colestid), colesevelam (Welchol), or combinations thereof or any combination thereof.

The compositions and methods can include use of one or more prokinetic agents at the same dosage as the recommended dosage, or in amounts less than the typical recommended dose for GERD. The prokinetic agents that can be used in the compositions and methods can include prokinetic agents such as bethanechol (Urecholine), metoclopramide (Reglan), domperidone (Motilium), cisapride (Propulsid), or combinations thereof or any combination thereof.

The compositions and methods can include use of one or more dopamine receptor antagonists at the same dosage as the recommended dosage, or in amounts less than the typical recommended dose for GERD. The dopamine receptor antagonists that can be used in the compositions and methods can include dopamine receptor antagonists such as domperidone (Motilium), metoclopramide (Reglan), or combinations thereof or any combination thereof.

The compositions can include one or more coating agents (protectants) at the same dosage as the recommended dosage, or in amounts less than the typical recommended dose for GERD. The coating agents (protectants) that can be used in the compositions and methods can include coating agents (protectants) such as sucralfate (Carafate), alginate (Gaviscon), hyaluronic acid plus chondroitin sulfate, or combinations thereof or any combination thereof.

The compositions can include one or more antibiotics at the same dosage as the recommended dosage, or in amounts less than the typical recommended dose for GERD. The antibiotics that can be used in the compositions and methods can include antibiotics such as penicillins, cephalosporins, aminoglycosides, fluoroquinolones, carbapenems, tetracyclines, doxycyclines, macrolides, erythromycins, azithromycins, polymyxins, or any combination thereof.

The compositions can include one or more probiotics at the same dosage as the recommended dosage, or in amounts less than the typical recommended dose for GERD. The probiotics that can be used in the compositions and methods can include probiotics such as products containing Lactobacillus, Bifidobacterium, Saccharomyces boulardii or any other useful bacteria or combination thereof, or any combination thereof.

The compositions can be administered or ingested once a day, or twice a day, or three times a day, or four times a day. In some cases, the compositions can be administered or ingested every day for one week, or for one month, or for two months, or for three months, or for six months, or for one year, or for two years, or for three years. In many cases the compositions can be administered or ingested every day indefinitely. The compositions may be administered in single or divided dosages. In some cases, the compositions are administered in the evening.

The compositions described herein can include a mixture of MIMOs as described herein. Inactive ingredients can be present in the compositions such a mannitol and some of the other components described herein. The mannitol is present in amounts too low to act as an osmotic laxative.

For example, the MIMOs can be present as about 50%, or about 60%, or about 65%, or about 70%, or about 75%, or about 80%, or about 85%, or about 90% or about 95%, or about 96%, or about 97%, or about 98%, or about 99% of the composition.

The compositions can include MIMOs in amounts of at least about 0.01 mg/kg to about 100 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. For example, the compositions can be administered or ingested in amounts of at least about 0.1 g, or at least about 0.25 g, or at least about 0.5 g, or at least about 0.7 g, or at least about 0.8 g, or at least about 0.9 g, or at least about 1.0 g, or at least about 1.1 g, or at least about 1.2 g. The unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

The amount administered will vary depending on various factors including, but not limited to, what types of compound(s), and/or other therapeutic agents are administered, the route of administration, the progression or lack of progression of the disease, the weight, the physical condition, the health, the age of the patient, whether prevention or treatment is to be achieved, and if the antigen or ligand is chemically modified. Such factors can be readily determined by the clinician employing animal models or other test systems that are available in the art.

Compounds and compositions thereof may be administered in a single dose, in multiple doses, in a continuous or intermittent manner, depending, for example, upon the recipient's physiological condition, whether the purpose of the administration is therapeutic or prophylactic, and other factors known to skilled practitioners. The administration of the compositions may be essentially continuous over a pre-selected period of time or may be in a series of spaced doses. Both local and systemic administration is contemplated.

In some cases, the compositions are formulated as liquid formulations. Alternatively, the MIMO compounds and other ingredients may be in powder form, obtained by aseptic isolation of sterile solid or by lyophilization from solution, for constitution with a suitable vehicle, e.g., sterile, pyrogen-free water, before use.

When the MIMOs are prepared for oral administration, they can be combined with a carrier. Such a carrier can be a pharmaceutically acceptable carrier, diluent or excipient. The compositions can be provided in the form of a unit dosage form. For oral administration, the MIMOs may be present as a powder, a granular formulation, a solution, a suspension, an emulsion or in a natural or synthetic polymer or resin for ingestion of the active ingredients from a chewing gum. The therapeutic agents may also be presented as a bolus, electuary or paste.

In some case, the compositions can be prepared for, and administered as, oral compositions. For example, tablets or caplets containing the compounds, and optionally a carrier can include buffering agents such as calcium carbonate, magnesium oxide and magnesium carbonate. Caplets and tablets can also include inactive ingredients such as cellulose (e.g., microcrystalline cellulose), pre-gelatinized starch, silicon dioxide (colloiodal), hydrogenated glucose syrup, polyol esters (mixture of mono, di, and tri esters), maltitol syrup, hydroxypropyl methyl cellulose (Hypromellose), sodium carboxymethylcellulose as-is and cross-linked (croscarmellose, disintegrant), magnesium or calcium stearate, microcrystalline cellulose, starch, talc, titanium dioxide, ascorbic acid and derivatives (e.g. ascorbyl palmitate), benzoic acid and derivatives (e.g. Na/K benzoate), sorbic acid and derivatives (e.g. Na/K sorbates), citric acid, tartaric acid, phosphoric acid, corn starch, mineral oil, benzyl alcohol, polypropylene glycol, sodium phosphate, zinc stearate, and the like. Hard or soft gelatin capsules, or gummies, containing at least one therapeutic agent of the invention can contain inactive ingredients such as gelatin, microcrystalline cellulose, surfactant, starch, talc, and titanium dioxide, and the like, as well as liquid vehicles such as polyethylene glycols (PEGs) and vegetable oil. Moreover, enteric-coated caplets or tablets containing one or more of the compounds of the invention are designed to resist disintegration in the stomach and dissolve in the more neutral to alkaline environment of the duodenum.

The compositions can also include antioxidants, surfactants, preservatives, film-forming, keratolytic or comedolytic agents, perfumes, flavorings and colorings. Antioxidants such as cysteine, N-acetylcysteine (NAC), propyl gallate, sodium or potassium metabisulfite, t-butylhydroquinone, 3,4-dihydroxybenzoic acid, benzoic acid esters (e.g. p-hydroxybenzoate, methyl, ethyl, or propylparaben), sesamol, guaiac resin, methionine, butylated hydroxyanisole (BHA), butylated hydroxytoluene (BHT), α-tocopherol (vitamin E) along with its derivatives (e.g., other tocopherols with four tocotrienols), and antioxidant syngergists, e.g. sodium or potassium edetate (tetrasodium EDTA, ethylenediaminetetracetic acid) and free or salt-forms of diethylenetriaminepentaacetic acid (DTPA) can be added.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present application, the preferred methods and materials are now described.

As utilized in accordance with the present disclosure, the following terms, unless otherwise indicated, shall be understood to have the following meanings:

“Brix”, also known as degrees Brix (symbol °Bx), refers to the sugar content of an aqueous solution. One degree Brix is 1 gram of sucrose in 100 grams of solution and represents the strength of the solution as percentage by weight (% w/w). Brix also accounts for dissolved salts, organic acids, and other solutes that increase the refractive index of the solution. As such, it is less useful as a quantitative measure of saccharide content in complex broth (fermentation mixtures) but is quite accurate with respect to the refined product. Thus, 1 degree brix=1 g refractive dry solids per 100 g of material. If the solution contains dissolved solids other than pure sucrose, then the °Bx only approximates the dissolved solid content. However, when the constituent components of the compositions to be compared are similar and/or within similar ranges, Brix values are reproducible and provide an approximation which, in this case, is an accurate (relative to true dry solids via evaporation) measurement of relative dry solids per each composition.

“Optical density” or “OD” refers to an estimation of cellular density in a fermentation. Typically used to determine the progress of a fermentation, it is determined via absorbance of light at 600 nm and may be referenced to dry cell mass.

“HPAEC-PAD” refers to a hyphenated instrumental analytical technique known as High Pressure Anion Exchange (HPAEC) liquid chromatography (ThermoDionex ICS-5000+) with a Pulsed Amperometric Detector (PAD). Under the scope of this work, this instrument is used solely for the high-resolution separation (ThermoDionex Carbopac PA-100, pH>12.5, acetate gradient elution) of sugar alcohols, mono and disaccharides, and oligosaccharides. Quantification is done via internal standard using L-arabinose and response factors relative to either the pure compound or to a purified maltodextrin of equivalent molecular weight.

“HPLC-RID” refers to a hyphenated instrumental analytical technique known as High Pressure Liquid Chromatography (HPLC, Agilent 1100) with a Refractive Index Detector (RID). Under the scope of this work, this instrument is used to separate (BioRad Aminex HPX-87H, 0.008N H₂SO₄ isocratic) and quantify organic (carboxylic) acids that result from bacterial fermentation. This instrument is also used to confirm DP, maltose, and mannitol. Quantification is done via external standard method vs. a mixed standard made from target compounds of known purity.

“SIP” refers to sterilized in place.

“SBF” refers to sterilization by filtration through a 0.2 μm membrane.

“CIP” refers to cleaned in place.

“DSP” refers to downstream processing.

“PRMXE” refers to filter cartridge series PRMXE or “Pur-Maxx E” dual-layer pleated polyethersulfone membrane sterilizing grade (SG) cartridges; 0.20 μm.

“Degree of polymerization”, or “DP”, refers to the number of monosaccharide sugar units in a given oligosaccharide.

“Oligosaccharides” refers to glycans of all kinds with DP of at least 3 and generally 10 or less.

“Molecular weight distribution,” or “MWD” refers to the mass-average molecular weight (Mw) of a distribution of oligosaccharides.

“Oligosaccharides” refers to glycans of all kinds, generally with a degree of polymerization (DP) greater than or equal to 3 and less than or equal to 18. “Maltosyl-isomaltooligosaccharides,” or MIMOs, refers to oligosaccharide, typically of less than 10-18 degrees of polymerization comprised of α-(1→6) linkages and terminated by an α-(1→4) glucosyl linkage. The α-(1→4) terminal group originates from maltose. Therefore, maltosyl-isomaltooligosaccharide or MIMO is produced by an acceptor reaction by maltose or other maltooligosaccharide. An example of a MIMO with a single maltosyl linkage [—O-α-(1,4)-] at the reducing end, and accordant with the panose core structure, DP 5 is maltosyl-isomaltotriose (technically isomaltotetrosyl-α-(1,4)-glucopyranose), which has the following chemical structure:

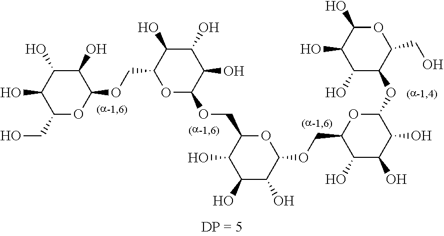 [SMILES: OC[C@H]1O[C@H](OC[C@H]2O[C@H](OC[C@H]3O[C@H](OC[C@H]4O[C@H](O[C@H]5[C@H](O)[C@@H](O)[C@@H](O)O[C@@H]5CO)[C@H](O)[C@@H](O)[C@@H]4O)[C@H](O)[C@@H](O)[C@@H]3O)[C@H](O)[C@@H](O)[C@@H]2O)[C@H](O)[C@@H](O)[C@@H]1O]

“Branched MIMO” refers to an oligosaccharide, specifically MIMO, of less than or equal to 10 degrees of polymerization comprised of α-(1-6) linkages terminated by an α-(1,4) glucosyl linkage and α-(1,2), α-(1,3) and/or α-(1,4) branches. Examples of a branched MIMO with glucose branching linkages at positions 1,2 and 1,3 and/or 1,4 have the following structures:

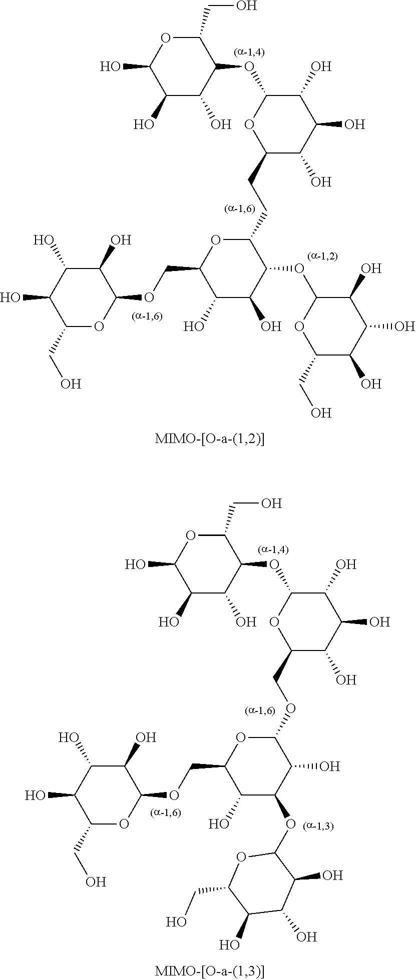

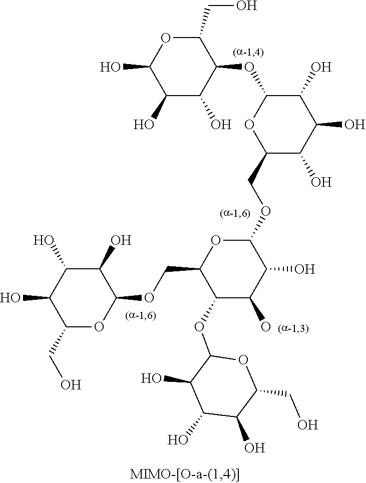 [SMILES: OC[C@H]1O[C@H](OC[C@H]2O[C@H](OC[C@H]3O[C@H](O[C@H]4[C@H](O)[C@@H](O)[C@@H](O)O[C@@H]4CO)[C@H](O)[C@@H](O)[C@@H]3O)[C@H](O)[C@@H](O)[C@@H]2OC2O[C@H](CO)[C@@H](O)[C@H](O)[C@H]2O)[C@H](O)[C@@H](O)[C@@H]1O]

“Dietary supplement” refers to a food, food ingredient, or food additive that produces a health benefit. Carbohydrates such as oligosaccharides can be dietary supplements.

“SAC” means a Strong Acid Cation exchange resin, typically one with sulfonic acid groups, i.e., a sulfuric acid equivalent. In some instances, SAC is referring to Purolite C-150S, which has a H capacity of 1.8 mol/L.

“WBA” means a Weak Base Anion exchange resin, typically one with tertiary amine groups, which are not stronger than the corresponding free base (pKa˜9.8). In this case, WBA is referring to Purolite A-133 which has a free-base capacity of 1.8 mol/L.

The Examples illustrate some of the experimental work performed in the development of the invention.

### Example 1: Materials and Methods

A study was performed to determine whether the test material, ISOT-101 (MIMO), was well-tolerated and altered the symptoms typically experienced by GERD patients. The MIMO contained 80-90 percent pure maltosyl-isomaltooligosaccharides.

The study began in March 2020 when a total of 110 subjects were screened for GERD using stringent eligibility criteria by the study coordinator and the principal investigator. Potentially confounding co-morbidities were excluded. The ISOT-101 (MIMO) was administered starting in June 2020. The subjects acted as their own controls and there was no placebo arm. All subjects had active symptoms as determined by verbal history and daily ReQuest (RQ) validated GERD questionnaire symptom scores above specified thresholds, or GERD symptoms at four or more days per week.

As of May 2021, 44 subjects had completed the following protocol. After one week of baseline screening (week 0), subjects took one sachet (1.4 cc or about 1 gram of ISOThrive MIMO every night at bedtime) of ISOT-101 daily for four additional weeks, reporting daily RQ questionnaires. Symptom evaluation for each included: RQ_GI: acid and abdominal (upper/lower) complaints, nausea; RQ_WSO: general well-being, sleep disturbances, and other complaints; and RQ_TOT: the sum of both. The Short Form 36 Health Survey validated questionnaire (see webpage physio-pedia.com/36-Item_Short_Form_Survey_(SF-36)) was used to assess quality of life. The SF-36 form was completed by subjects on day seven of week zero as well as on each day seven per week during the study duration. The subjects had to report any usage of GERD medications such as PPIs, H2 antagonists, and acids throughout the study, as well as any changes in their other medications. The primary endpoint was tolerability, defined as ending on week four with a request symptom score and a SF-36 score that was at or better than the baseline week zero score (determined as >0% reduction in weekly mean RQ symptom scores as % from baseline to week 4). Statistical analysis was done using the Wilcoxon signed-rank test with multiple comparison correction. In addition, a comparison was made of the number of days without acid complaints per week, or the heartburn-free days per week, for week four compared to the week zero baseline.

### Protocol Summary:

Patient Population: 61 adult men and women, self-reported as having GERD, were screened and 35 were offered ISOT-101 as a trial for tolerability. Of these, 29 were enrolled and 24 completed the study. Approximately half of these had been taking ISOT-101 previously for non-GERD symptoms and all had been off ISOT-101 for at least 6 months. 33% of subjects reported taking PPI upon enrollment, 8% reported taking H2 blockers. All subjects reported to have been previously diagnosed with mild to moderate GERD, and that they had not been diagnosed with BE or EAC.

Trial Design. A qualifying questionnaire was emailed via surveygizmo.com (ReQuest Qualifying, 2019). Only those subjects with symptomatic GERD were included. The subjects were instructed to take ISOT-101 daily for 30 days (1 g every night just before bed, making it the last thing swallowed before lying down for the night). To measure tolerability, subjects were emailed a modified ReQuest questionnaire via surveygizmo.com to fill out at time 0 (baseline before starting ISOT-101) (ReQuest Enrollment, 2019), and then once per week for 4 consecutive weeks while taking ISOT-101 daily (ReQuest Weekly, 2019). The GERD ReQuest symptom questionnaires were modified to have subjects report symptoms weekly as opposed to daily. ReQuest is a validated questionnaire designed to measure daily symptoms of GERD by patient reported outcomes (Bardhan, 2004; Monnikes, 2004; Bardhan, 2007; ReQuest Validated Form, 2004). The ReQuest Total Score is the sum of the ReQuest GI symptom score and the ReQuest WSO score, which includes wellness, sleep and other QOL issues.

Results: ISOT-101 appeared to be quite tolerable. Only one subject experienced an increase in symptom score at end of study vs. baseline. No adverse events were reported. Scores were compared by week relative to baseline.

Conclusion/Discussion: The intent of this study was to explore the tolerability of ISOT-101 in a population of GERD patients. With respect to all 7 symptom-related questions asked, all symptom scores improved.

A deficiency in this study were the weak screening parameters which relied upon a self-reported prior GERD diagnosis, which resulted in four (4) subjects included who clearly did not have GERD. Nevertheless, the data collected was very strong and appears to confirm tolerability of ISOT-101 in GERD patients.

### Detailed Protocol:

The test material was ISOT-101, containing approximately 90% pure maltosyl-isomalto-oligosacchride (MIMO) prebiotic syrup produced by bacterial fermentation/bio-conversion of sucrose and maltose. It was taken 1 g daily at bedtime.

Subjects were previously diagnosed with GERD and fell into one of the following four groups:

1. Symptomatic subjects currently not taking PPI therapy and who are naive to PPIs.

2. Symptomatic subjects currently not taking PPI therapy who were responsive to prior PPI therapy (either QD or BID) and who have been off PPIs for at least four weeks.

3. Symptomatic subjects currently taking PPI therapy (either QD or BID) who are partial responders to PPIs.

4. 10 symptomatic subjects who have had no benefit from PPIs and have not taken PPIs for at least 4 weeks.

All subjects had active GERD symptoms as determined by the ReQuest validated GERD specific questionnaire and/or GERD symptoms at least 4 days per week. The study was designed to have each subject serve as their own control so there will be no placebo arm. Each subject received ISOT-101 according to the protocol. Recruitment for subjects with GERD was by referrals from GI specialists, primary care physicians, and general advertising. Each candidate was given information as to how to contact the study coordinator to enroll in the study. The goal was to assess 110 subjects after the Screening Phase, including the 10 subjects who did not respond to PPIs. All subjects were screened per the eligibility criteria delineated in this protocol by the study coordinator. The study was done remotely. There was communication but no in person contact or visits with the study coordinator.

Two phases were designed into this study. Phase 1 was the Screening Phase (SP). Phase 2 was the Tolerability Phase (TP). Subjects were instructed to take a daily GERD symptom questionnaire, the ReQuest Short Version, daily throughout the study. At five timepoints, specific scores were established for comparison and statistical analysis. These timepoints were: (1) SP7—baseline on current therapy (if any) without ISOT-101; (2) TP7—1 week on both current therapy (if any) and ISOT-101; (3) TP14—2 weeks on both current therapy (if any) and ISOT-101; (4) TP21—3 weeks on both current therapy (if any) and ISOT-101; (5)) TP28—4 weeks on both current therapy (if any) and ISOT-101. At each of these timepoints, subjects were supposed to complete the ReQuest Long Version and the SF-36 Health Survey QoL validated questionnaires. They did need not to complete the ReQuest Short Version on days that the ReQuest Long Version is required. Subjects reported any usage of all GERD medications (PPI, H-2 antagonists, antacid) throughout their study participation.

### Eligibility Inclusion Criteria:

1. English-speaking male and female adults between the ages of 25 and 75, inclusive

2. Body mass index (BMI)<35 and >19

3. Either 3.a. or 3.b. or both, based on the Screening Phase ReQuest Short and Long Version responses:
a. At least one incidence of (a) acid complaints; (b) upper abdominal/stomach complaints and RQ Total score >3.37 or RQ-GI score >0.95
b. Four days or more of combined incidence of (a) acid complaints: (b) upper abdominal/stomach complaints.

4. History (minimum of three months) of GERD symptoms as defined in #3 above

5. Must be on stable doses of medications, if any, prescribed for chronic conditions other than GERD

6. If female, must be on an active contraceptive measure or have male partner(s) with suitable protective measures

7. Have access to a computer/tablet/phone with internet access and active e-mail account in order to complete electronic surveys daily throughout study participation

8. Ability and willingness to give consent to participate in study

### Exclusion Criteria:

1. Significant comorbidities that are not medically stable

2. History of scleroderma, diabetes, Barrett's esophagus, esophageal cancer, esophageal stricture, or esophageal scarring (fibrosis)

3. Known hiatal hernia >2 cm

4. Positive diagnosis for Helicobacter pylori (HP) or has had an HP infection within 45 days of study entry

5. History of surgery or endoscopic treatment including fundoplication and dilation for esophageal stricture

6. History of gastric surgery (except for endoscopic removal of benign polyps) or bariatric surgery

7. BMI>=35, BMI<=19

8. History of diseases that have symptoms that may be confused with GERD, such as eosinophilic esophagitis, angina, gastritis, esophageal spasm, rumination, or other conditions involving the mouth, throat, chest or abdomen

9. Active history of nicotine use, cannabis use or alcohol abuse (as defined by: greater than 14 drinks/week or 4 drinks/day for men, 7 drinks/week or 3 drinks/day for women). Has used any tobacco, nicotine or cannabis products in the last 6 months or has abused alcohol in the last 6 months

10. Taking any excluded medications listed in the protocol (e.g. metformin, antibiotics within the prior 6 months.)

11. If female, was pregnant, lactating, or intending to become pregnant before, during or within 4 weeks after participating in this study or intending to donate ova during such time period

12. Primary sleep disorder including sleep apnea, restless leg syndrome, or insomnia

13. Colon prep within 30 days prior to study entry

14. Use of any investigational product within 90 days prior to study entry

15. Participation in another investigation (clinical trial) during the course of this study

16. Participation in a rigorous weight loss program or have any planned changes in diet or lifestyle, such as getting married, change in residence, change in job, or other highly stressful event

17. Diagnosis of inflammatory bowel disease, irritable bowel syndrome, or chronic diarrhea (mild chronic constipation was allowed)

18. Employment (or relative of an employee) or involvement in any way with ISOThrive Inc.

19. An investigator, key study personnel or first degree relative of anyone involved with the study

20. Other conditions or situations that, in the investigator's opinion, may put the subject at significant risk, may confound the study results, or may interfere significantly with the subject's participation in the study

### Enrollment

An adequate number of individuals were needed to be screened during the Screening Phase, using disease specific validated questionnaires to yield approximately 110 qualified subjects to enter and likely complete the Tolerability Phase of the study. Recruitment involved primary care physicians and/or gastroenterologists who would make IRB approved study information available to patients who are symptomatic for GERD. Such patients included those partial responders on PPI therapy; those who responded to PPIs, but had previously stopped their PPI therapy for at least 4 weeks; those who were naive to PPI therapy, and 10 subjects that have had no benefit from PPIs and had not been on PPI therapy for at least 4 weeks. Deviations from the inclusion and exclusion criteria were not allowed so as not to jeopardize the scientific integrity of the study, regulatory acceptability, or subject safety. Therefore, adherence to the criteria as specified in the protocol was needed.

All subjects were required to sign an IRB-approved informed consent or e-consent that complies with the requirements of both 21 CRF Part 50 and Health Insurance Portability and Accountability Act (HIPAA) before entering the study.

The duration of the study is defined for each subject as the date a signed written informed consent (or e-consent) is provided through Tolerability Phase Day 28. Total participation in study lasted up to 8 weeks. A comparable gender distribution was sought for the final analysis, and the study sought a ratio of male to female (or female to male) subjects completing the Tolerability Phase at 60% of the total.

### Concomitant Medications and Washout Periods

GERD medications: Subjects currently taking PPI therapy (either QD or BID) who were partial responders to PPIs were allowed to continue taking PPIs throughout the study as needed. Subjects not taking PPIs prior to being enrolled could also take PPIs as needed. Antacids or H-2 antagonists were allowed to be taken regularly or intermittently as needed throughout the conduct of this study. All GERD-related medications taken by the subject were recorded daily via the daily electronic survey.

Prebiotic and/or probiotic supplements: Enrolled subjects stopped taking all prebiotic and/or probiotic supplements 2 weeks prior to entering the Screening Phase of the study. Subjects refrained from taking prebiotic or probiotic supplements through Tolerability Phase Day 28.

Sleep medications: Enrolled subjects taking medications for sleep disorders were on a stable dose at time of consent and continue at said dose through Tolerability Phase Day 28.

Antibiotics: Subjects must not have taken antibiotics within 6 months prior to signing of consent or at any time during their participation in the study. If antibiotics were prescribed to the subject during study participation, subject were withdrawn from study and discontinued daily ISOT-101 immediately.

All study product sachets (used and unused) were returned to Investigator for product accountability.

### 5.0 Subject Study Flow Process

No study-related activities were performed and no subject data were collected prior to completion of the informed consent process.

### Screening Phase (SP):

To enter the SP, subjects must have previously been diagnosed with GERD, be symptomatic on their current therapy or lack of therapy, and fell into one of the 4 study groups noted in Section 4.0 above. The SP lasted for 7 days. Subjects who did not qualify at any point during the SP were not allowed to move into the Tolerability Phase (TP). The TP lasted 28 days beginning with first dose of ISOT-101.

### Day 1 (Initial Encounter):—

After consent has been obtained, the study coordinator will assist the subject with the following activities:
Review eligibility criteria
Gather medical history
Record all medications and supplements
Record reported height and weight
   If the subject qualifies:
Establish access to electronic surveys and educate subject on how to access the surveys and complete the questionnaires
Complete daily ReQuest Short Version electronically
Record daily medication use electronically

### Days 2-6

Complete daily ReQuest Short Version electronically
Record daily medication use electronically

### Day 7

Complete ReQuest Long Version electronically
Complete SF-36 health questionnaire electronically
Record daily medication use electronically

Following Day 7 of SP, the average of the daily ReQuest Short Version scores were used to establish a baseline score to confirm that the subject is experiencing GERD symptoms. Any subject who had a ReQuest Short Version (RQ) average score of >=3.37 (90 percentile) or ReQuest GI (RQ-GI) average score >=0.95 was eligible to transition to the Tolerability Phase. The ReQuest Short Version SP1-SP7 average and SF-36 served as a baseline. Investigator verified eligibility prior to study contacting subject.

Once eligibility was verified by the PI, the study coordinator informed the subjects and confirms that a subject wished to continue in the study. Once confirmed, study coordinator:
Reviewed concomitant medications
Collected any adverse events
Reviewed calendar for remainder of study
Provided instructions for daily dosing of ISOT-101
Confirmed mailing address
Arranged for shipment of 30-day supply of ISOT-101 to the subject

There may have been up to 7 calendar days between SP Day 7 and TP Day 1 depending on length of time to ship/receive ISOT-101.

### Tolerability Phase (TP)

Subjects took their current GERD therapy as follows:
If on PPI therapy QD or BID, then the PPI were to be taken as the subject customarily had taken it, prior to beginning the study.
If on an H-2 antagonist or antacid, then the subject could continue these medications as needed throughout the study.

All GERD medications and other medications were recorded daily via the electronic survey.
ISOT-101 was taken daily at bedtime (one sachet per day). This will be the last thing swallowed prior to bedtime (no rinsing, and after brushing teeth)

### Day 1 (Phone Call or E-mail)

Confirm receipt of ISOT-101
Review concomitant medications
Collect adverse events
Complete daily ReQuest Short Version electronically
Record medications taken electronically
Record 1st dose of ISOT-101 electronically

### Days 2-13

Complete daily ReQuest Short Version electronically
Record daily medications electronically
Record daily dose of ISOT-101 electronically

### Day 14 (Phone Call or E-mail)

Review concomitant medications
Collect adverse events
Complete daily ReQuest Short Version electronically
Record medications taken electronically
Record dose of ISOT-101 electronically

### Days 15-27

Complete daily ReQuest Short Version electronically
Record daily medications electronically
Record daily dose of ISOT-101 electronically

### Day 28 (Phone Call or E-mail)

Collect adverse events
Complete ReQuest Long Version electronically
Complete SF-36 health questionnaire electronically
Record medications taken electronically
Record dose of ISOT-101 electronically
Place used and unused ISOT-101 sachets in provided Ziploc bag and return in postage-paid envelope
Answer additional questions.
   As the primary endpoint, tolerability of ISOT-101 was assessed via changes in the ReQuest Short Version GERD symptom scores (RQ, RQ-GI and RQ-WSO) and SF-36 scores. These were assessed for all subjects as well as for the sub-group of subjects that completed all 4 weeks of the protocol. Changes in ReQuest symptom scores, or SF-36 scores, between baseline and each of the 4 weeks of data were the basis of this assessment. Further evaluation included comparison of ISOT-101 tolerability in the 4 subject sub-groups using these time points.

The safety endpoint of the study was to ensure that any adverse events that are reported or observed during the study are appropriately recorded.

### Example 2: MIMOs Reduce Acid Indigestion in Subjects with GERD

This Example illustrates that maltosyl-isomaltooligosaccharides (ISOT-101) increase the number of acid-free days per month in GERD subjects.

Appendices B-D describe the results in detail. The following is a summary of the results. results were separated into four subgroups based on mean weekly changes in the ReQuest (RQ) validated GERD questionnaire symptom scores, on the general well-being, sleep disturbances, and other complaints (RQ_WSO) scores, and on the total scores. Such subgrouping were used to show whether a placebo could compare favorably with treatment by maltosyl-isomaltooligosaccharides (ISOT-101) and to evaluate whether one prebiotic (e.g. MIMOs) would work well for everyone.

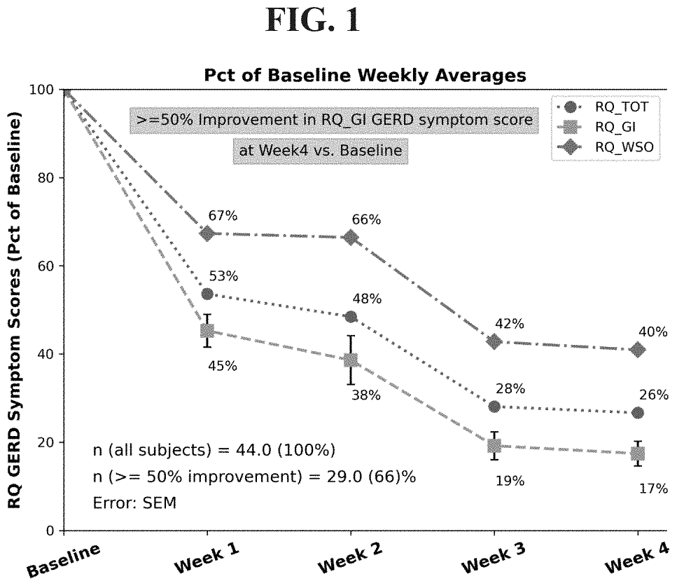

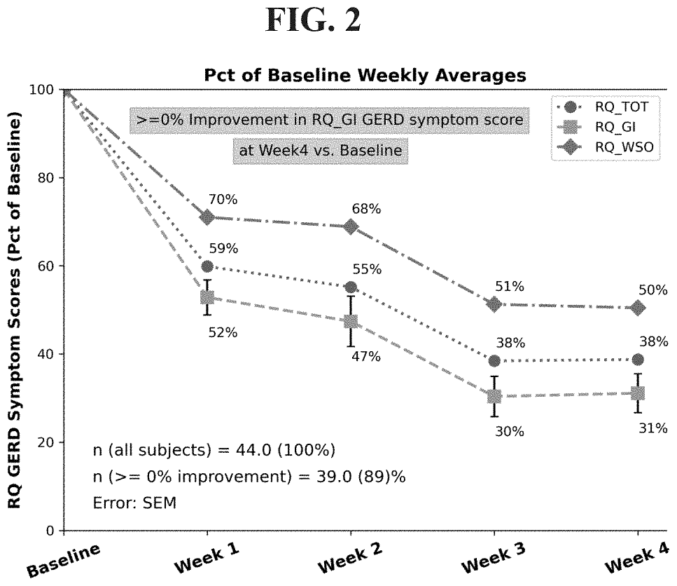

In summary, the average weekly RQ_GI symptom scores and RQ_WSO scores were as follows:
a) All Subjects (100% of subjects n=44) reduced [GI: 100% to 48%, WSO: 100% to 58%],
b) All Tolerating Subjects (89% of subjects n=39) reduced [GI: 100% to 31%, WSO: 100% to 50%] (FIG. 2)
c) Partial or Better Responders (84% of subjects n=37) reduced [GI: 100% to 28%, WSO: 100% to 48%]
d) Strong Responders (66% of subjects n=29) reduced [GI: 100% to 17%, WSO: 100% to 40%] by week 4 (FIG. 1)
   All comparisons are between mean week zero scores and mean week four scores. FIGS. 1 and 2 show weekly averages and symptom scores as well. Nausea occurred in 2 patients (5%). Five (5) of 44 (110%) had worsening of RQ_GI (gastrointestinal symptoms). Four (4) of 44 (9%) had worsening of RQ_TOT (total scores). There were no ISOT-101-related adverse events. Data for two subgroups are shown in FIGS. 1 and 2, but additional data is shown in Appendix B-D (see also Appendix A).

Subgroup A represented 100 percent of the subjects who finished the tolerability phase (i.e. 44 subjects). From week zero to week four the GI scores for Subgroup A were reduced from 100 percent at week zero to 48 percent at week four. The general well-being, sleep disturbances, and other complaint (RQ_WSO) scores for Subgroup A were reduced from 100 percent at week zero to 58 percent at week four. Total scores for Subgroup A were reduced from 100 to 50 percent. All weeks in all categories were significantly different from week zero with p-values less than 0.001.

Subgroup B represented 89 percent of the subjects—those who tolerated ISOT-101. Five subjects either did not improve or got worse and are not included in this group. Hence, Subgroup B includes 39 subjects. FIG. 2 illustrates the tolerability of Subgroup B. From week zero to week four, the GI scores for Subgroup B were reduced from one hundred to 31 percent. The general well-being, sleep disturbances, and other complaint (RQ_WSO) scores for Subgroup B were reduced from 100 to 50 percent, while the Total scores for Subgroup B were reduced from 100 to 38 percent. All weeks in all categories were significantly different from week zero with p-values less than 0.001.

Subgroup C represents 84 percent of the subjects—those who are partial or better responders to ISOT-101. Subgroup C includes 37 subjects. From week zero to week four the GI scores for Subgroup C were reduced from 100 percent to 28 percent. The general well-being, sleep disturbances, and other complaint (RQ_WSO) scores for Subgroup B were reduced from reduced from 100 percent at week zero to 48 percent at week four, while the Total scores were reduced from 100 to 35 percent. All weeks in all categories were significantly different from week zero with a p-value less than 0.001.

Subgroup D represents 66 percent of the subjects—those who improved by at least 50 percent. Subgroup D includes 29 subjects. These subjects improved to the point that they were asymptomatic as defined by the study request thresholds. These subjects corresponds to the strong responder subgroup in FIG. 1. From week zero to week four the GI scores for Group D were reduced from one hundred to seventeen percent. WSO scores were reduced for Group D subjects from one hundred percent to forty percent while the Total scores for Group D were reduced from 100 to 26 percent. All weeks for all categories evaluated for Group D were significantly different from week zero with a p-value less than 0.001.

During baseline week zero, the average number of days without acid complaints per week, or heartburn-free days was 0.33. The average number of heartburn free days during week four was 2.4. Hence, the number of heartburn free days increased significantly during the four week study. The number of heartburn free days each week as the study progressed was statistically significantly different from the number of heartburn free days observed during week zero, with a p-value less than 0.001. These results indicate that taking ISOT-101 can increase the number of heartburn free days by more than eight days per month.

ISOT-101 is a non-digestible, non-absorbable prebiotic carbohydrate. It is well tolerated and it significantly increases the frequency of heartburn free days in subjects with GERD. It also reduces the severity of symptoms in GERD subjects and improves their quality of life. These results show that ISOT-101 can be an effective treatment for GERD either in combination with other GERD medications or as a stand-alone therapy. It also may inhibit the progression of GERD to Barrett's to adenocarcinoma, as the inflammation that is characteristic of this progression appears to be the same type of immune cytokine cascade that dysbiosis creates.

### Example 3: Subjects with Greater Acid Severity Benefit Most from MIMOs

Data obtained using the methods described in Example 1 were evaluated using a simple difference in acid-free days between week 0 and week 4 of MIMO treatment to determine which GERD subjects benefited the most. Several variables (subject medication, demographics, scores) were added to a simple model to see variable(s) could predict improvement in subject acid-free days.

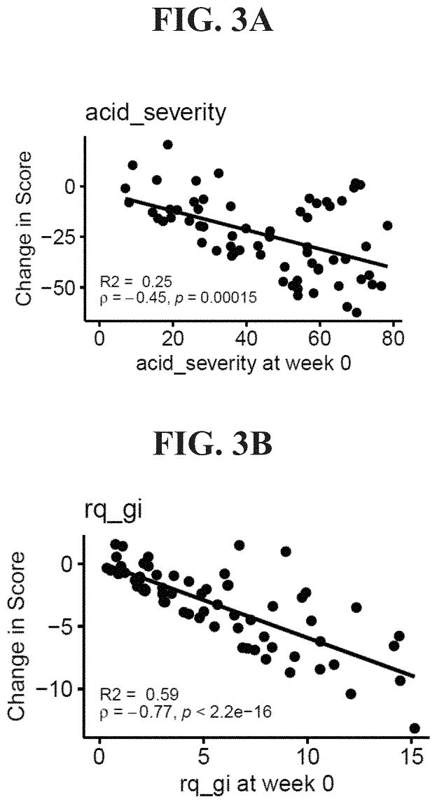

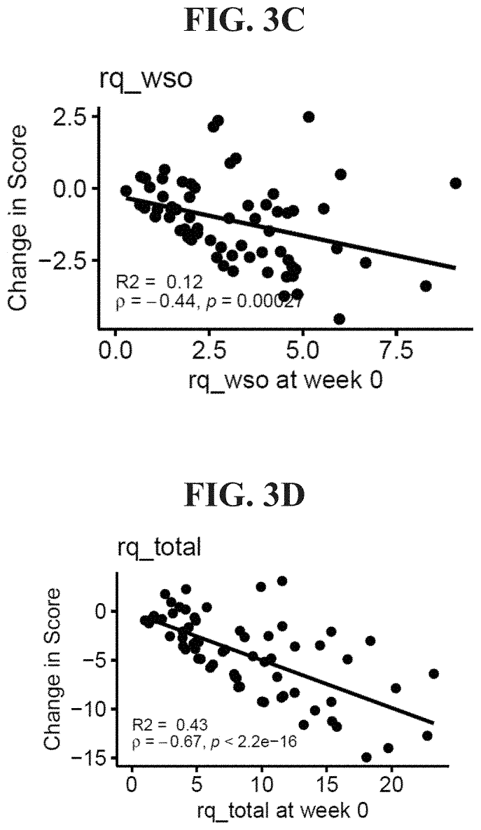

Appendix D describes data analysis and results in more detail, but as summarized shown in FIG. 3A-3D, the subjects with the more severe ERD symptoms exhibited the most improvement when taking MIMOs.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements describe and summarize various embodiments of the invention according to the foregoing description in the specification.

### Statements:

1. A composition comprising maltosyl-isomaltooligosaccharides and one or more proton pump inhibitors, one or more potassium-competitive acid blockers, one or more H-2 antagonists, one or more antacids, one of more bile acid sequestrants, one or more prokinetic agents, one or more dopamine receptor antagonists, one or more coating agents (protectants), one or more antibiotics, one or more probiotics, or a combination thereof.
2. The composition of statement 1, comprising maltosyl-isomaltooligosaccharides and one or more proton pump inhibitors and/or one or more potassium-competitive acid blockers.
3. The composition of statement 1, comprising maltosyl-isomaltooligosaccharides and one or more H-2 antagonists.
4. The composition of statement 1, comprising maltosyl-isomaltooligosaccharides and one or more antacids.
5. The composition of statement 1, comprising maltosyl-isomaltooligosaccharides and one or more bile acid sequestrants.
6. The composition of statement 1, comprising maltosyl-isomaltooligosaccharides and one or more prokinetic agents.
7. The composition of statement 1, comprising maltosyl-isomaltooligosaccharides and one or more dopamine receptor antagonists.
8. The composition of statement 1, comprising maltosyl-isomaltooligosaccharides and one or more coating agents (protectants).
9. The composition of statement 1, comprising maltosyl-isomaltooligosaccharides and one or more antibiotics.
10. The composition of statement 1, comprising maltosyl-isomaltooligosaccharides and one or more probiotics.
11. The composition of any one of statements 1-10, further comprising a carrier.
12. The composition of any one of statements 1-11, wherein the one or more proton pump inhibitor is Omeprazole (Prilosec®), Esomeprazole (Nexium®), Lansoprazole (Prevacid®), Rabeprazole (AcipHex®), Pantoprazole (Protonix®), Dexlansoprazole (Dexilant®), Zegerid®, or any combination thereof.
13. The composition of any one of statements 1-12, wherein the one or more potassium-competitive acid blocker is Revaprazan (Revanex), Vonoprazan (Takecab), Tegoprazan, or any combination thereof.
14. The composition of any one of statements 1-13, wherein the one or more H-2 antagonist is Famotidine (e.g., Pepcid AC, Pepcid Oral), Cimetidine (e.g., Tagamet, Tagamet HB), Ranitidine (e.g., Zantac, Zantac 75, Zantac Efferdose, Zantac injection, and Zantac Syrup), Nizatidine Capsules (e.g., Axid AR, Axid Capsules, Nizatidine Capsules), or combinations thereof.
15. The composition of any one of statements 1-14, wherein the one or more antacids comprise calcium carbonate, aluminum hydroxide, magnesium hydroxide, sodium bicarbonate, or combinations thereof.
16. The composition of any one of statements 1-15, wherein the one or more bile acid sequestrant is cholestyramine (Questran, Prevalite), colestipol (Colestid), colesevelam (Welchol), or any combinations thereof.
17. The composition of any one of statements 1-16, wherein the one or more prokinetic agent is bethanechol (Urecholine), metoclopramide (Reglan), domperidone (Motilium), cisapride (Propulsid), or any combinations thereof.
18. The composition of any one of statements 1-17 wherein the one or more dopamine receptor antagonist is metoclopramide (Reglan), domperidone (Motilium), or any combinations thereof.
19. The composition of any one of statements 1-18 wherein the one or more coating agents (protectants) is sucralfate (Carafate), alginate (Gaviscon), hyaluronic acid plus chondroitin sulfate, or any combination thereof.
20. The composition of any one of statements 1-19 wherein the one or more antibiotics is penicillins, cephalosporins, aminoglycosides, fluoroquinolones carbapenems, tetracyclines, doxycyclines, macrolides, erythromycins, azithromycins, and polymyxins, or any combinations thereof.
21. The combination of any one of statements 1-20 wherein the one or more probiotics is products containing Lactobacillus, Bifidobacterium, Saccharomyces boulardii or any other useful bacteria or any combination thereof.
22. The composition of any one of statements 1-21, wherein the maltosyl-isomaltooligosaccharides have a mass average molecular weight distribution of about 730 to 900 daltons, and wherein at least 40% of the maltosyl-isomaltooligosaccharides in the composition have a degree of polymerization (DP) of 3 or more.
23. The composition of any one of statements 1-22, where the maltosyl-isomaltooligosaccharides have no more than about 18 glucose units.
24. The composition of any one of statements 1-23, where the maltosyl-isomaltooligosaccharides have a maltose unit at the reducing end.
25. The composition of any one of statements 1-24, with less than 2%/brix isomaltose; less than 5%/brix glucose; less than 5%/brix sucrose; less than 4%/brix fructose; less than 7%/brix lactate; or less than 8%/brix maltose.
26. The composition of any one of statements 1-25, with 3% or more than 3%/brix mannitol.
27. The composition of any one of statements 1-26, with less than 4%/brix glycerol.
28. The composition of any one of statements 1-27, with less than 20%/brix MIMO-DP3.
29. The composition of any one of statements 1-28, with less than 30%/brix MIMO-DP4.
30. The composition of any one of statements 1-29, with more than 18%/brix MIMO-DP5.
31. The composition of any one of statements 130, with more than 10%/brix MIMO-DP6.
32. The composition of any one of statements 1-31, with more than 1%/brix MIMO-7.
33. The composition of any one of statements 1-32, with more than 0.5%/brix MIMO-DP8.
34. The composition of any one of statements 1-33, with more than 0.1%/brix MIMO-DP9.
35. The composition of any one of statements 1-34, as a concentrated solution.
36. The composition of any one of statements 1-35, dried as a powder.
37. The composition of any one of statements 1-36, aliquoted into individual servings for human or animal consumption.
38. The composition of any one of statements 1-37, aliquoted into individual serving of about 0.25 ml to about 10 ml.
39. A method for treating gastroesophageal reflux disease (GERD) in a subject, comprising administering to the subject, or consuming by the subject, a composition comprising maltosyl-isomaltooligosaccharides with a mass average molecular weight distribution of about 730 to 900 daltons, wherein at least 40% of the maltosyl-isomaltooligosaccharides in the composition have a degree of polymerization (DP) of 3 or more.
40. The method of statement 39, wherein the composition further comprises one or more proton pump inhibitors, one or more potassium-competitive acid blockers, one or more H-2 antagonists, one or more antacids, one or more bile acid sequestrants, one or more prokinetic agents, one or more dopamine receptor antagonists, one or more coating agents (protectants), one or more antibiotics, one or more probiotics, or a combination thereof.
41. The method of statement 39 or 40, wherein the composition further comprises a carrier.
42. The method of any one of statements 40 or 41, wherein the one or more proton pump inhibitors is Omeprazole (Prilosec®), Esomeprazole (Nexium®), Lansoprazole (Prevacid®), Rabeprazole (AcipHex®), Pantoprazole (Protonix®), Dexlansoprazole (Dexilant®), Zegerid®, or any combination thereof.
43. The method of any one of statements 40-42, wherein the one or more potassium-competitive acid blockers is Revaprazan (Revanex), Vonoprazan (Takecab), Tegoprazan, or any combination thereof.
44. The method of any one of statements 40-43, wherein the one or more H-2 antagonists is Famotidine (e.g., Pepcid AC, Pepcid Oral), Cimetidine (e.g., Tagamet, Tagamet HB), Ranitidine (e.g., Zantac, Zantac 75, Zantac Efferdose, Zantac injection, and Zantac Syrup), Nizatidine Capsules (e.g., Axid AR, Axid Capsules, Nizatidine Capsules), or combinations thereof.
45. The method of any one of statements 40-44, wherein the one or more antacids comprises calcium carbonate, aluminum hydroxide, magnesium hydroxide, sodium bicarbonate, or combinations thereof
46. The method of any one of statements 40-45, wherein the one or more bile acid sequestrants is cholestyramine (Questran, Prevalite), colestipol (Colestid), colesevelam (Welchol), or any combinations thereof.
47. The method of any one of statements 40-46 wherein the one or more prokinetic agents is bethanechol (Urecholine), metoclopramide (Reglan), domperidone (Motilium), cisapride (Propulsid), or combinations thereof.
48. The method of any one of statements 40-47 wherein the one or more dopamine receptor antagonists is metoclopramide (Reglan), domperidone (Motilium), or any combinations thereof.
49. The method of any one of statements 40-48 wherein the one or more coating agents (protectants) is sucralfate (Carafate), alginate (Gaviscon), hyaluronic acid plus chondroitin sulfate, or any combinations thereof.
50. The method of any one of statements 40-49 wherein the one or more antibiotics is penicillins, cephalosporins, aminoglycosides, fluoroquinolones, carbapenems, tetracyclines, doxycyclines, macrolides, erythromycins, azithromycins, polymyxins, or any combinations thereof.
51. The method of any one of statements 40-50 wherein the one or more probiotics is products containing Lactobacillus, Bifidobacterium, Saccharomyces boulardii or any other useful bacteria, or any combination thereof.
52. The method of any one of statements 39-51, wherein the composition comprises one or more proton pump inhibitors in an amount equal to or less than the recommended daily dosage.
53. The method of any one of statements 39-52, wherein the composition comprises the proton pump inhibitor in an amount less than a daily dosage of 20 mg, in an amount less than a daily dosage of 15 mg, in an amount less than a daily dosage of 10 mg, in an amount less than a daily dosage of 7 mg, in an amount less than a daily dosage of 5 mg, in an amount less than a daily dosage of 4 mg, in an amount less than a daily dosage of 3 mg, in an amount less than a daily dosage of 2 mg, or in an amount less than a daily dosage of 1 mg.
54. The method of any one of statements 39-53, wherein the composition comprises one or more potassium-competitive acid blockers in an amount equal to or less than the recommended daily dosage.
55. The method of any one of statements 39-54, wherein the composition comprises one or more potassium-competitive acid blockers in an amount less than a daily dosage of 150 mg, in an amount less than a daily dosage of 100 mg, in an amount less than a daily dosage of 75 mg, in an amount less than a daily dosage of 50 mg, in an amount less than a daily dosage of 25 mg, in an amount less than a daily dosage of 20 mg, in an amount less than a daily dosage of 15 mg, in an amount less than a daily dosage of 10 mg, in an amount less than a daily dosage of 7 mg, in an amount less than a daily dosage of 5 mg, in an amount less than a daily dosage of 4 mg, in an amount less than a daily dosage of 3 mg, in an amount less than a daily dosage of 2 mg, or in an amount less than a daily dosage of 1 mg.
56. The method of any one of statements 39-55, wherein the composition comprises one or more H-2 antagonists in an amount equal to or less than the recommended daily dosage.
57. The method of any one of statements 39-56, wherein the composition comprises one or more H-2 antagonists in an amount less than a daily dosage of 800 mg, in an amount less than a daily dosage of 700 mg, in an amount less than a daily dosage of 600 mg, in an amount less than a daily dosage of 500 mg, in an amount less than a daily dosage of 400 mg, in an amount less than a daily dosage of 300 mg, in an amount less than a daily dosage of 200 mg, in an amount less than a daily dosage of 100 mg, in an amount less than a daily dosage of 70 mg, in an amount less than a daily dosage of 60 mg, in an amount less than a daily dosage of 50 mg, in an amount less than a daily dosage of 40 mg, in an amount less than a daily dosage of 30 mg, or in an amount less than a daily dosage of 20 mg.
58. The method of any one of statements 39-57, wherein the composition comprises one or more antacids in an amount equal to or less than the recommended daily dosage.
59. The method of any one of statements 39-58, wherein the composition comprises one or more antacids in an amount less than a daily dosage of 4 g, in an amount less than a daily dosage of 3 g, in an amount less than a daily dosage of 2 g, in an amount less than a daily dosage of 1 g, in an amount less than a daily dosage of 800 mg, in an amount less than a daily dosage of 700 mg, in an amount less than a daily dosage of 600 mg, in an amount less than a daily dosage of 500 mg, in an amount less than a daily dosage of 400 mg, in an amount less than a daily dosage of 100 mg, in an amount less than a daily dosage of 75 mg, in an amount less than a daily dosage of 50 mg, in an amount less than a daily dosage of 30 mg, or in an amount less than a daily dosage of 20 mg.
60. The method of any one of statements 39-59, wherein the composition comprises one or more bile salt sequestrants in an amount equal to or less than the recommended daily dosage.
61. The method of any one of statements 39-60, wherein the composition comprises one or more bile acid sequestrants in an amount less than a daily dosage of 8 g, in an amount less than a daily dosage of 7 g, in an amount less than a daily dosage of 6 g, in an amount less than a daily dosage of 5 g, in an amount less than a daily dosage of 4 g, in an amount less than a daily dosage of 3 g, in an amount less than a daily dosage of 2 g, in an amount less than a daily dosage of 1 g, in an amount less than a daily dosage of 500 mg, in an amount less than a daily dosage of 100 mg, in an amount less than a daily dosage of 75 mg, in an amount less than a daily dosage of 50 mg, in an amount less than a daily dosage of 30 mg, or in an amount less than a daily dosage of 20 mg.
62. The method of any one of statements 39-61, wherein the composition comprises one or more prokinetic agents in an amount equal to or less than the recommended daily dosage.
63. The method of any one of statements 39-62, wherein the composition comprises one or more prokinetic agents in an amount less than a daily dosage of 40 mg, in an amount less than a daily dosage of 30 mg, in an amount less than a daily dosage of 20 mg, or in an amount less than a daily dosage of 10 mg.
64. The method of any one of statements 39-63, wherein the composition comprises one or more dopamine receptor antagonists in an amount equal to or less than the recommended daily dosage.
65. The method of any one of statements 39-64, wherein the composition comprises one or more dopamine receptor antagonists in an amount less than a daily dosage of 40 mg, in an amount less than a daily dosage of 30 mg, in an amount less than a daily dosage of 20 mg, or in an amount less than a daily dosage of 10 mg.
66. The method of any one of statements 39-65, wherein the composition comprises one or more coating agents (protectants) in an amount equal to or less than the recommended daily dosage.
67. The method of any one of statements 39-66, wherein the composition comprises one or more coating agents (protectants) in an amount less than a daily dosage of 4 g, in an amount less than a daily dosage of 3 g, in an amount less than a daily dosage of 2 g, in an amount less than a daily dosage of 1 g, in an amount less than a daily dosage of 500 mg, in an amount less than a daily dosage of 100 mg, in an amount less than a daily dosage of 75 mg, in an amount less than a daily dosage of 50 mg, in an amount less than a daily dosage of 30 mg, or in an amount less than a daily dosage of 20 mg.
68. The method of any one of statements 39-67, wherein the composition comprises one or more antibiotics in an amount equal to or less than the recommended daily dosage.
69. The method of any one of statements 39-68, wherein the composition comprises one or more antibiotics in an amount less than a daily dosage of 2 g, in an amount less than a daily dosage of 1 g, in an amount less than a daily dosage of 500 mg, in an amount less than a daily dosage of 100 mg, in an amount less than a daily dosage of 75 mg, in an amount less than a daily dosage of 50 mg, in an amount less than a daily dosage of 30 mg, or in an amount less than a daily dosage of 20 mg.
70. The method of any one of statements 39-69, wherein the composition comprises one or more probiotics in an amount equal to or less than the recommended daily dosage.
71. The method of any one of statements 39-70, wherein the composition comprises one or more probiotics in an amount less than a daily dosage of 10 billion units, in an amount less than a daily dosage of 9 billion units, in an amount less than a daily dosage of 8 billion units, in an amount less than a daily dosage of 7 billion units, in an amount less than a daily dosage of 6 billion units, in an amount less than a daily dosage of 5 billion units, in an amount less than a daily dosage of 4 billion units, in an amount less than a daily dosage of 3 billion units, in an amount less than a daily dosage of 2 billion units, in an amount less than a daily dosage of 1 billion units, in an amount less than a daily dosage of 500 million units, in an amount less than a daily dosage of 100 million units.
72. The method of any one of statements 39-71, which reduces the symptoms of GERD.
73. The method of any one of statements 39-72, which reduces the frequency of GERD symptoms.
74. The method of any one of statements 39-73, wherein the GERD symptoms comprise sensations of burning in the throat, chest pains, difficulty swallowing, regurgitation, regurgitating acid, sensations of a lump in the throat, chronic coughs, laryngitis, asthma, sleep disruption, or combinations thereof.
75. The method of any one of statements 39-74, which reduces the dosage of proton pump inhibitors or the symptoms of GERD by at least 10%, at least 20%, at least 30%, at least 40%, at least 50%, at least 60%, at least 70%, at least 80%, at least 90%, or at least 95%.
76. The method of any one of statements 39-75, which reduces the dosage of potassium-competitive acid blockers or the symptoms of GERD by at least 10%, at least 20%, at least 30%, at least 40%, at least 50%, at least 60%, at least 70%, at least 80%, at least 90%, or at least 95%.
77. The method of any one of statements 39-76, which reduces the dosage of H-2 antagonists or the symptoms of GERD by at least 10%, at least 20%, at least 30%, at least 40%, at least 50%, at least 60%, at least 70%, at least 80%, at least 90%, or at least 95%.
78. The method of any one of statements 39-77, which reduces the dosage of antacids or the symptoms of GERD by at least 10%, at least 20%, at least 30%, at least 40%, at least 50%, at least 60%, at least 70%, at least 80%, at least 90%, or at least 95%.
79. The method of any one of statements 39-77, which reduces the dosage of bile acid sequestrants or the symptoms of GERD by at least 10%, at least 20%, at least 30%, at least 40%, at least 50%, at least 60%, at least 70%, at least 80%, at least 90%, or at least 95%.
80. The method of any one of statements 39-78, which reduces the dosage of prokinetic agents or the symptoms of GERD by at least 10%, at least 20%, at least 30%, at least 40%, at least 50%, at least 60%, at least 70%, at least 80%, at least 90%, or at least 95%.
81. The method of any one of statements 39-79, which reduces the dosage of dopamine receptor antagonists or the symptoms of GERD by at least 10%, at least 20%, at least 30%, at least 40%, at least 50%, at least 60%, at least 70%, at least 80%, at least 90%, or at least 95%.
82. The method of any one of statements 39-80, which reduces the dosage of coating agents (protectants) or the symptoms of GERD by at least 10%, at least 20%, at least 30%, at least 40%, at least 50%, at least 60%, at least 70%, at least 80%, at least 90%, or at least 95%.
83. The method of any one of statements 39-81, which reduces the dosage of antibiotics and/or the symptoms of GERD by at least 10%, at least 20%, at least 30%, at least 40%, at least 50%, at least 60%, at least 70%, at least 80%, at least 90%, or at least 95%.
84. The method of any one of statements 39-82, which reduces the dosage of probiotics or the symptoms of GERD by at least 10%, at least 20%, at least 30%, at least 40%, at least 50%, at least 60%, at least 70%, at least 80%, at least 90%, or at least 95%.
85. The method of any one of statements 39-84, which reduces the frequency of proton pump inhibitor administration and/or the frequency of GERD symptoms by at least one day per week, or at least two days per week, or at least three days per week, or at least four days per week, or at least five days per week, or at least six days per week.
86. The method of any one of statements 39-85, which reduces the frequency of potassium-competitive acid blocker administration and/or the frequency of GERD symptoms by at least one day per week, or at least two days per week, or at least three days per week, or at least four days per week, or at least five days per week, or at least six days per week.
87. The method of any one of statements 39-86, which reduces the frequency of H-2 antagonist administration and/or the frequency of GERD symptoms by at least one day per week, or at least two days per week, or at least three days per week, or at least four days per week, or at least five days per week, or at least six days per week.
88. The method of any one of statements 39-87, which reduces the frequency of antacid administration and/or the frequency of GERD symptoms by at least one day per week, or at least two days per week, or at least three days per week, or at least four days per week, or at least five days per week, or at least six days per week.
89. The method of any one of statements 39-88, which reduces the frequency of bile acid sequestrants administration and/or the frequency of GERD symptoms by at least one day per week, or at least two days per week, or at least three days per week, or at least four days per week, or at least five days per week, or at least six days per week.
90. The method of any one of statements 39-89, which reduces the frequency of prokinetic agent administration and/or the frequency of GERD symptoms by at least one day per week, or at least two days per week, or at least three days per week, or at least four days per week, or at least five days per week, or at least six days per week.
91. The method of any one of statements 39-90, which reduces the frequency of domperidone receptor antagonist administration and/or the frequency of GERD symptoms by at least one day per week, or at least two days per week, or at least three days per week, or at least four days per week, or at least five days per week, or at least six days per week.
92. The method of any one of statements 39-91, which reduces the frequency of coating agent (protectant) administration and/or the frequency of GERD symptoms by at least one day per week, or at least two days per week, or at least three days per week, or at least four days per week, or at least five days per week, or at least six days per week.
93. The method of any one of statements 39-92, which reduces the frequency of antibiotic administration and/or the frequency of GERD symptoms by at least one day per week, or at least two days per week, or at least three days per week, or at least four days per week, or at least five days per week, or at least six days per week.
94. The method of any one of statements 39-93, which reduces the frequency of probiotic administration and/or the frequency of GERD symptoms by at least one day per week, or at least two days per week, or at least three days per week, or at least four days per week, or at least five days per week, or at least six days per week.
95. The method of any one of statements 39-94, wherein subjects suffering from GERD (e.g., who generally take proton pump inhibitors daily) need to take proton pump inhibitors only six times per week, only five times per week, only four times per week, only three times per week, only two times per week, only once per week, or no times per week.
96. The method of any one of statements 39-95, wherein subjects suffering from GERD (e.g., who generally take potassium-competitive acid blocker daily) need to take potassium-competitive acid blocker only six times per week, only five times per week, only four times per week, only three times per week, only two times per week, only once per week, or no times per week.
97. The method of any one of statements 39-96, wherein subjects suffering from GERD (e.g., who generally take H-2 antagonists daily) need to take H-2 antagonists only six times per week, only five times per week, only four times per week, only three times per week, only two times per week, only once per week, or no times per week.
98. The method of any one of statements 39-97, wherein subjects suffering from GERD (e.g., who generally take antacids daily) need to take antacids only six times per week, only five times per week, only four times per week, only three times per week, only two times per week, only once per week, or no times per week.
99. The method of any one of statements 39-98, wherein subjects suffering from GERD (e.g., who generally take bile acid sequestrants daily) need to take bile acid sequestrants only six times per week, only five times per week, only four times per week, only three times per week, only two times per week, only once per week, or no times per week.
100. The method of any one of statements 39-99, wherein subjects suffering from GERD (e.g., who generally take prokinetic agents daily) need to take prokinetic agents only six times per week, only five times per week, only four times per week, only three times per week, only two times per week, only once per week, or no times per week.
101. The method of any one of statements 39-100, wherein subjects suffering from GERD (e.g., who generally take dopamine receptor antagonists daily) need to take dopamine antagonists only six times per week, only five times per week, only four times per week, only three times per week, only two times per week, only once per week, or no times per week.
102. The method of any one of statements 39-101, wherein subjects suffering from GERD (e.g., who generally take coating agents (protectants) daily) need to take coating agents (protectants) only six times per week, only five times per week, only four times per week, only three times per week, only two times per week, only once per week, or no times per week.
103. The method of any one of statements 39-102, wherein subjects suffering from GERD (e.g., who generally take antibiotics daily) need to take antacids no times per week.
104. The method of any one of statements 39-103, wherein subjects suffering from GERD (e.g., who generally take probiotics daily) need to take probiotics only six times per week, only five times per week, only four times per week, only three times per week, only two times per week, only once per week, or no times per week.
105. The method of any one of statements 39-104, comprising any of the following administration regimens:
   a. Start taking one or more proton pump inhibitors, potassium-competitive acid blockers (P-CABs), H-2 antagonists, antacids, bile acid sequestrants, prokinetic agents, dopamine receptor antagonists to achieve a baseline of improvement, then start taking maltosyl-isomaltooligosaccharides to increase improvement;
   b. Start taking both maltosyl-isomaltooligosaccharides with one or more proton pump inhibitors, potassium-competitive acid blockers (P-CABs), H-2 antagonists, antacids, bile acid sequestrants, prokinetic agents, dopamine receptor antagonists;
   c. Start as described in either (a) or (b) above, and then stop taking the one or more proton pump inhibitors, potassium-competitive acid blockers (P-CABs), H-2 antagonists, antacids, bile acid sequestrants, prokinetic agents, dopamine receptor antagonists, or stop any combination thereof;
   d. Taking the maltosyl-isomaltooligosaccharides at night, or at night after brushing, and after eating and drinking has ceased;
   e. Taking the maltosyl-isomaltooligosaccharides in the morning;
   f Taking the maltosyl-isomaltooligosaccharides at any time during the day;
   g. Taking the maltosyl-isomaltooligosaccharides in any of regimens (a)-(f) at a dosage of 1 gram per dose, or 2 gram per dose, or more than 2 grams per dose; or
   h. Any of regimens (a)-(g).

The specific compositions and methods described herein are representative, exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications can be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a compound” or “an oligosaccharide” or “a maltose” includes a plurality of such compounds, oligosaccharides, or maltose sugars, and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

What is claimed:

1. A composition comprising maltosyl-isomaltooligosaccharides and one or more proton pump inhibitors, one or more potassium-competitive acid blockers, one or more H-2 antagonists, one or more antacids, one or more bile acid sequestrants, one or more prokinetic agents, one or more dopamine receptor antagonists, one or more coating agents (protectants), one or more antibiotics, one or more probiotics, or a combination thereof, wherein the maltosyl-isomaltooligosaccharides have a mass average molecular weight distribution of about 730 to 900 daltons, and wherein at least 40% of the maltosyl-isomaltooligosaccharides in the composition have a degree of polymerization (DP) of 3 or more.

2. A method for treating gastroesophageal reflux disease (GERD) in a subject, comprising administering to the subject, or consuming by the subject, a composition comprising maltosyl-isomaltooligosaccharides with a mass average molecular weight distribution of about 730 to 900 daltons, wherein at least 40% of the maltosyl-isomaltooligosaccharides in the composition have a degree of polymerization (DP) of 3 or more.

3. The method of claim 2, wherein the composition further comprises one or more proton pump inhibitors.

4. The method of claim 3, which reduces the dosage of proton pump inhibitors or the symptoms of GERD by at least 10%, at least 20%, at least 30%, at least 40%, at least 50%, at least 60%, at least 70%, at least 80%, at least 90%, or at least 95%.

5. The method of claim 2, which reduces the frequency of proton pump inhibitor administration and/or the frequency of GERD symptoms by at least one day per week, or at least two days per week, or at least three days per week, or at least four days per week, or at least five days per week, or at least six days per week.

6. The method of claim 2, wherein the composition further comprises one or more potassium-competitive acid blockers.

7. The method of claim 6, which reduces the dosage of the one or more potassium-competitive acid blockers or the symptoms of GERD by at least 10%, at least 20%, at least 30%, at least 40%, at least 50%, at least 60%, at least 70%, at least 80%, at least 90%, or at least 95%.

8. The method of claim 2, which reduces the frequency of potassium-competitive acid blockers administration and/or the frequency of GERD symptoms by at least one day per week, or at least two days per week, or at least three days per week, or at least four days per week, or at least five days per week, or at least six days per week.

9. The method of claim 2, wherein the composition further comprises one or more H-2 antagonists.

10. The method of claim 2, which reduces the dosage of the one or more H-2 antagonists or the symptoms of GERD by at least 10%, at least 20%, at least 30%, at least 40%, at least 50%, at least 60%, at least 70%, at least 80%, at least 90%, or at least 95%.

11. The method of claim 2, which reduces the frequency of H-2 antagonist administration and/or the frequency of GERD symptoms by at least one day per week, or at least two days per week, or at least three days per week, or at least four days per week, or at least five days per week, or at least six days per week.

12. The method of claim 2, wherein the composition further comprises one or more antacids.

13. The method of claim 2, which reduces the dosage of the one or more antacids or the symptoms of GERD by at least 10%, at least 20%, at least 30%, at least 40%, at least 50%, at least 60%, at least 70%, at least 80%, at least 90%, or at least 95%.

14. The method of claim 2, which reduces the frequency of antacid administration and/or the frequency of GERD symptoms by at least one day per week, or at least two days per week, or at least three days per week, or at least four days per week, or at least five days per week, or at least six days per week.

15. The method of claim 2, wherein the composition further comprises one or more bile acid sequestrants.

16. The method of claim 2, which reduces the dosage of the one or more bile acid sequestrants or the symptoms of GERD by at least 10%, at least 20%, at least 30%, at least 40%, at least 50%, at least 60%, at least 70%, at least 80%, at least 90%, or at least 95%.

17. The method of claim 2, which reduces the frequency of bile acid sequestrant administration and/or the frequency of GERD symptoms by at least one day per week, or at least two days per week, or at least three days per week, or at least four days per week, or at least five days per week, or at least six days per week.

18. The method of claim 2, wherein the composition further comprises one or more prokinetic agents.

19. The method of claim 2, which reduces the dosage of the one or more prokinetic agents or the symptoms of GERD by at least 10%, at least 20%, at least 30%, at least 40%, at least 50%, at least 60%, at least 70%, at least 80%, at least 90%, or at least 95%.

20. The method of claim 2, which reduces the frequency of prokinetic agent administration and/or the frequency of GERD symptoms by at least one day per week, or at least two days per week, or at least three days per week, or at least four days per week, or at least five days per week, or at least six days per week.

21. The method of claim 2, wherein the composition further comprises one or more dopamine receptor antagonists.

22. The method of claim 2, which reduces the dosage of the one or more dopamine receptor antagonists or the symptoms of GERD by at least 10%, at least 20%, at least 30%, at least 40%, at least 50%, at least 60%, at least 70%, at least 80%, at least 90%, or at least 95%.

23. The method of claim 2, which reduces the frequency of dopamine receptor antagonist administration and/or the frequency of GERD symptoms by at least one day per week, or at least two days per week, or at least three days per week, or at least four days per week, or at least five days per week, or at least six days per week.

24. The method of claim 2, wherein the composition further comprises one or more coating agents (protectants).

25. The method of claim 2, which reduces the dosage of the one or more coating agents (protectants) or the symptoms of GERD by at least 10%, at least 20%, at least 30%, at least 40%, at least 50%, at least 60%, at least 70%, at least 80%, at least 90%, or at least 95%.

26. The method of claim 2, which reduces the frequency of coating agents (protectants) administration and/or the frequency of GERD symptoms by at least one day per week, or at least two days per week, or at least three days per week, or at least four days per week, or at least five days per week, or at least six days per week.

27. The method of claim 2, wherein the composition further comprises one or more antibiotics.

28. The method of claim 2, which reduces the dosage of the one or more antibiotics or the symptoms of GERD by at least 10%, at least 20%, at least 30%, at least 40%, at least 50%, at least 60%, at least 70%, at least 80%, at least 90%, or at least 95%.

29. The method of claim 2, which reduces the frequency of antibiotic administration and/or the frequency of GERD symptoms by at least one day per week, or at least two days per week, or at least three days per week, or at least four days per week, or at least five days per week, or at least six days per week.

30. The method of claim 2, wherein the composition further comprises one or more probiotics.

31. The method of claim 2, which reduces the dosage of the one or more probiotics or the symptoms of GERD by at least 10%, at least 20%, at least 30%, at least 40%, at least 50%, at least 60%, at least 70%, at least 80%, at least 90%, or at least 95%.

32. The method of claim 2, which reduces the frequency of probiotic administration and/or the frequency of GERD symptoms by at least one day per week, or at least two days per week, or at least three days per week, or at least four days per week, or at least five days per week, or at least six days per week.
